(19) United States
(12) Patent Application Publication
(10) Pub. No.: US 2026/0320927 A1
(43) Pub. Date: Oct. 15, 2026

(54) METHODS AND APPARATUS TO REDUCE ANXIETY WITH MIXED REALITY

(71) Applicant: Abbott Laboratories, Abbott Park, IL (US)

(72) Inventors: Miguel Carrazza Morales, Chicago, IL (US); Rahul Avasthy, Gurnee, IL (US); Jeffrey Ditusa, Elmhurst, IL (US); Erin Banks, Western Springs, IL (US); Deanna Cardone, Abbott Park, IL (US); James Donohoe, Abbott Park, IL (US)

(21) Appl. No.: 19/120,146

(22) PCT Filed: Oct. 16, 2023
(86) PCT No.: PCT/US2023/076985

§ 371 Date: Apr. 10, 2025

Related U.S. Application Data
(60) Provisional application No. 63/416,827, filed on Oct. 17, 2022.
(60) Provisional application No. 63/579,726, filed on Aug. 30, 2023.

(51) Int. Cl. G06T 19/00; G02B 27/01; G06F 3/01; G06F 3/16; G06T 7/20; G06T 7/70; G06T 13/40; G16H 40/63

(52) U.S. Cl. CPC G06T 19/006; G02B 27/017; G06F 3/013; G06F 3/167; G06T 7/20; G06T 7/70; G06T 13/40; G16H 40/63; G02B 2027/0178

(57) ABSTRACT

Methods and apparatus to reduce anxiety using mixed reality are disclosed herein. An example method of reducing anxiety in a person during a blood collection process includes providing a mixed reality headset to a person prior to collecting blood from the person. The method includes initiating a mixed reality program on the mixed reality headset. The mixed reality program causes a display device to display a mixed reality environment with one or more virtual objects on glasses of the headset, determines a gaze direction of the eyes of the person, and causes a change in one or more of the virtual objects in the mixed reality environment based on the gaze direction. The method further includes collecting blood from the person while the person is exposed to the mixed reality program.

### FIELD OF THE DISCLOSURE

This disclosure relates generally to anxiety reduction and, more particularly, to methods and apparatus to reduce anxiety with mixed reality.

### BACKGROUND

Blood collection for medical testing or for donation causes anxiety in some people. This anxiety makes some people reluctant to donate blood.

### BRIEF DESCRIPTION OF THE DRAWINGS

FIG. 1 illustrates an example environment in which a person wears an example mixed reality headset during an example blood collection process.

FIG. 2 illustrates an example physical implementation and an example block diagram of the example headset of FIG. 1.

FIGS. 3A-3L show example views through the example headset of FIGS. 1 and 2 of an example mixed reality environment from the perspective of the person wearing the example headset.

FIG. 4 is a flowchart of an example method to reduce anxiety during a blood collection process.

FIG. 5 is a flowchart representative of example machine readable instructions and/or example operations that may be executed, instantiated, and/or performed by example programmable circuitry to implement an example mixed reality program or application of FIG. 2.

FIG. 6 is a block diagram of an example processing platform including programmable circuitry structured to execute the example, instantiate, and/or perform machine readable instructions and/or perform the example operations of FIG. 5 to implement the example mixed reality program or application of FIG. 2.

FIG. 7 is a block diagram of an example implementation of the programmable circuitry of FIG. 6.

FIG. 8 is a block diagram of another example implementation of the programmable circuitry of FIG. 6.

FIG. 9 is a block diagram of an example software/firmware/instructions distribution platform (e.g., one or more servers) to distribute software, instructions, and/or firmware (e.g., corresponding to the example machine readable instructions of FIG. 5) to client devices associated with end users and/or consumers (e.g., for license, sale, and/or use), retailers (e.g., for sale, re-sale, license, and/or sub-license), and/or original equipment manufacturers (OEMs) (e.g., for inclusion in products to be distributed to, for example, retailers and/or to other end users such as direct buy customers).

In general, the same reference numbers will be used throughout the drawing(s) and accompanying written description to refer to the same or like parts. The figures are not necessarily to scale.

### DETAILED DESCRIPTION

Blood banks and blood donation centers are constantly seeking blood donations from people. Donated blood is used for blood transfusions in a wide range of circumstances, such as for organ transplant patients, blood disorder patients, and/or during operations such as surgeries involving serious injuries, child birth, cancer treatments, heart surgery, etc. Donated blood is also used during emergency situations such as in the wake of natural disaster events.

A blood collection process is typically administered by a medical professional such as a nurse, doctor, or phlebotomist. During the blood collection process, the phlebotomist inserts a needle into a vein in a person's (donor's) arm. Blood is then drawn from the person's vein and collected into a bag or other container. The blood collection process typically lasts about 8-10 minutes (but can sometimes be longer or shorter). Typically, about 0.5 liters (L) of whole blood is collected during a blood collection process. During the blood collection process, the person (donor) is typically sitting, laying down, and/or in a reclined position. When a sufficient amount of blood has been collected, the phlebotomist removes the needle from the person's arm.

Many people experience trypanophobia (commonly referred to as needle-phobia), needle anxiety, and/or are otherwise fearful or uncomfortable with needles. This prevents or limits people from donating blood and/or participating in other medical procedures that involve use of a needle.

Disclosed herein are example methods and apparatus to help calm or relax a person during an anxiety-inducing process or situation, such as during a blood collection process or other medical or dental procedure. Example methods and apparatus disclosed herein can entertain the person and/or otherwise direct the person's attention away from the blood collection process. This helps reduce their anxiety, fear, and/or otherwise calms the person during the blood collection process. As such, people are less fearful of a blood donation operation and will therefore be more likely to donate blood. Therefore, the example methods and apparatus disclosed herein can also increase donor engagement, satisfaction, and retention.

An example method disclosed herein includes exposing the person to a mixed reality immersive experience during an anxiety-inducing situation such as, for example, a blood collection process. In some examples, the person wears a headset with a display, also known as a head-mounted display or HMD, that provides the person with the mixed reality experience. Mixed reality is a type of immersive experience sometimes referred to more broadly as extended reality (XR). XR also includes virtual reality and augmented reality.

Virtual reality (VR) is a technology that utilizes software and a headset with a display that is worn by a person. The software causes the display to present a virtual reality environment and can track the user's location and viewpoint within the virtual reality environment. The person can move their head to view different areas within the virtual reality environment and interact with objects in the virtual reality environment. In VR, the display completely covers the person's eyes, blocking out the person's surroundings. Therefore, the person can only see the virtual reality environment presented on the display.

Augmented reality (AR) is a computer-based technology that combines the real world with the digital world. AR is typically used on electronic displays such as, for example, smartphone screens, where the screen displays a live view from a camera and overlays digital objects on the live view. AR systems sometime use computer vision to detect objects, planes, and/or faces, and then overlays digital objects on these planes and surfaces. AR is commonly seen with filters that augment faces.

Mixed reality (MR) is a combination of VR and AR that blends the real world with computer-generated elements and/or digital information so that a person in a mixed reality experience sees both physical objects and virtual objects in the same space. MR utilizes a headset or glasses with transparent glass that enables the person to see their surroundings as normal. The MR software analyzes and stores the locations of structures and objects in the surrounding environment. The headset displays digital objects on the glasses, such that the objects appear to be in the real-world surroundings or environment. With MR, the software stores the locations of the digital objects in relation to the real-world surroundings, so the digital objects appear in the same locations of the real-world surroundings when the person looks around at their surroundings. In other words, the digital objects appear fixed relative to one or more objects or surfaces in the real-world surroundings. Therefore, unlike AR, the software does not need to reanalyze the live view and constantly perform object recognition. MR also enables the person to interact with the digital objects.

Examples methods and apparatus disclosed herein utilize MR to entertain and/or distract the person during an anxiety-inducing situation such as, for example, a blood collection process. Further, the MR experience not only distracts the person from certain visual cues (e.g., the needle in their arm) that may trigger or cause anxiety, but the MR experience may partially or completely block the visual cues from sight. Also, in some examples, MR is advantageous because the headset or glasses can be worn on the person's head while the blood collection process occurs. This enables the person to keep their hands and arms free and stationary during the procedure. Also, the MR headset or glasses have transparent glass. During the blood collection process, the phlebotomist monitors the person for adverse effects such as a vasovagal event, dizziness, and/or spatial awareness problems. The phlebotomist monitors the person's eyes to determine if there is an event occurring and/or an impending onset of such an event. Therefore, using a MR headset with transparent glass enables the phlebotomist to continue to monitor the person for a possible event or adverse effect even while the person's is engaged with the MR experience.

In some examples disclosed herein the mixed reality environment includes scenes or objects from nature, such as a plants and trees. In some examples, scenes or objects from nature are calming, which helps the person relax during the blood collection process. In other examples, the mixed reality environment can include other types of environments or scenes. In some examples, the mixed reality environment is interactive. For example, the person may use their eyes to control one or more features in the mixed reality environment. This helps focus the person's attention on the mixed reality environment instead of the blood collection process.

While example methods and apparatus disclosed herein are described in connection with blood donation operations, the example methods an apparatus disclosed herein can also be used in connection with other types of medical procedures involving a needle, such as, for example, blood collection for medical testing, vaccination, and/or insertion of an IV. Examples disclosed herein can also be used in connection with medical procedures not involving a needle, but nonetheless may cause the person anxiety or discomfort, such as, for example, during insertion of a catheter. Examples disclosed herein can be used for any medical or dental procedure for, for example, people who have hemophobia (fear of blood), iatrophobia (fear of doctors or medical tests), and/or dentophobia (fear of dentist). In addition, examples disclosed herein may be used in other scenarios such as, for example, during travel on an airplane for people who are aerophobic or otherwise anxious on airplanes. Thus, the example methods and apparatus disclosed herein can be used in connection with any type of procedure, situation, and/or environment where a person may be fearful or have anxiety. The example methods and apparatus disclosed herein can be used to help calm or relax the person during the procedure, situation, and/or environment. Therefore, the example methods and apparatus disclosed herein improve safety to not only the person but also to the phlebotomist and other persons administering the procedure.

Unless specifically stated otherwise, descriptors such as “first,” “second,” “third,” etc., are used herein without imputing or otherwise indicating any meaning of priority, physical order, arrangement in a list, and/or ordering in any way, but are merely used as labels and/or arbitrary names to distinguish elements for ease of understanding the disclosed examples. In some examples, the descriptor “first” may be used to refer to an element in the detailed description, while the same element may be referred to in a claim with a different descriptor such as “second” or “third.” In such instances, it should be understood that such descriptors are used merely for identifying those elements distinctly within the context of the discussion (e.g., within a claim) in which the elements might, for example, otherwise share a same name.

As used herein, the phrase “in communication,” including variations thereof, encompasses direct communication and/or indirect communication through one or more intermediary components, and does not require direct physical (e.g., wired) communication and/or constant communication, but rather additionally includes selective communication at periodic intervals, scheduled intervals, aperiodic intervals, and/or one-time events.

As used herein, “programmable circuitry” is defined to include (i) one or more special purpose electrical circuits (e.g., an application specific circuit (ASIC)) structured to perform specific operation(s) and including one or more semiconductor-based logic devices (e.g., electrical hardware implemented by one or more transistors), and/or (ii) one or more general purpose semiconductor-based electrical circuits programmable with instructions to perform specific function(s) and/or operation(s) and including one or more semiconductor-based logic devices (e.g., electrical hardware implemented by one or more transistors). Examples of programmable circuitry include programmable microprocessors such as Central Processor Units (CPUs) that may execute first instructions to perform one or more operations and/or functions,

Field Programmable Gate Arrays (FPGAs) that may be programmed with second instructions to cause configuration and/or structuring of the FPGAs to instantiate one or more operations and/or functions corresponding to the first instructions, Graphics Processor Units (GPUs) that may execute first instructions to perform one or more operations and/or functions, Digital Signal Processors (DSPs) that may execute first instructions to perform one or more operations and/or functions, XPUs, Network Processing Units (NPUs) one or more microcontrollers that may execute first instructions to perform one or more operations and/or functions and/or integrated circuits such as Application Specific Integrated Circuits (ASICs). For example, an XPU may be implemented by a heterogeneous computing system including multiple types of programmable circuitry (e.g., one or more FPGAs, one or more CPUs, one or more GPUs, one or more NPUs, one or more DSPs, etc., and/or any combination(s) thereof), and orchestration technology (e.g., application programming interface(s) (API(s)) that may assign computing task(s) to whichever one(s) of the multiple types of programmable circuitry is/are suited and available to perform the computing task(s).

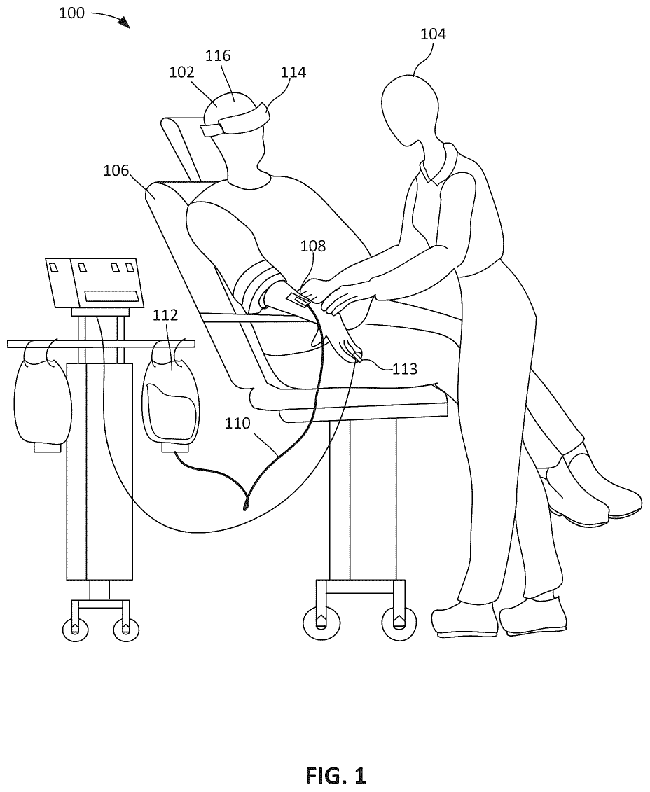

Turning to the figures, FIG. 1 illustrates an example environment 100 in which the example methods and apparatus disclosed herein can be implemented. In the example environment 100, a person 102 is donating blood, such as at a blood donation center or facility. The person 102 may also be referred to as a patient. In FIG. 1, the blood collection process is being administered by a phlebotomist or other medical professional 104.

In a typical blood donation operation, the person 102 arrives at the blood donation center and fills out paperwork. The person 102 is then directed to sit, lay, and/or recline in a chair 106. In other examples, the person 102 may remain standing. Then the blood collection process occurs. In the illustrated example, the phlebotomist 104 inserts a needle 108 into a vein in the person's 104 arm. However, in other examples, the needle 108 can be inserted into another location on the person's body. Blood from the needle 108 is routed by a tube 110 to a bag 112, where the blood is collected. After a sufficient amount of blood is collected and/or a certain amount of time has passed, the phlebotomist 104 removes the needle 108 and the blood collection process is complete.

In some examples, one or more sensors 113 (e.g., a pulse oximeter) is/are connected to the person 102 to obtain one or more vital signs (e.g., blood pressure, heart rate, temperature, blood oxygen level, etc.) of the person 102. While the blood is being collected, the phlebotomist 104 monitors the one or more vital signs of the person 102. Additionally or alternatively, the phlebotomist 104 monitors the general disposition of the person 102, such as monitoring the person's eyes to ensure the person 102 is awake and coherent.

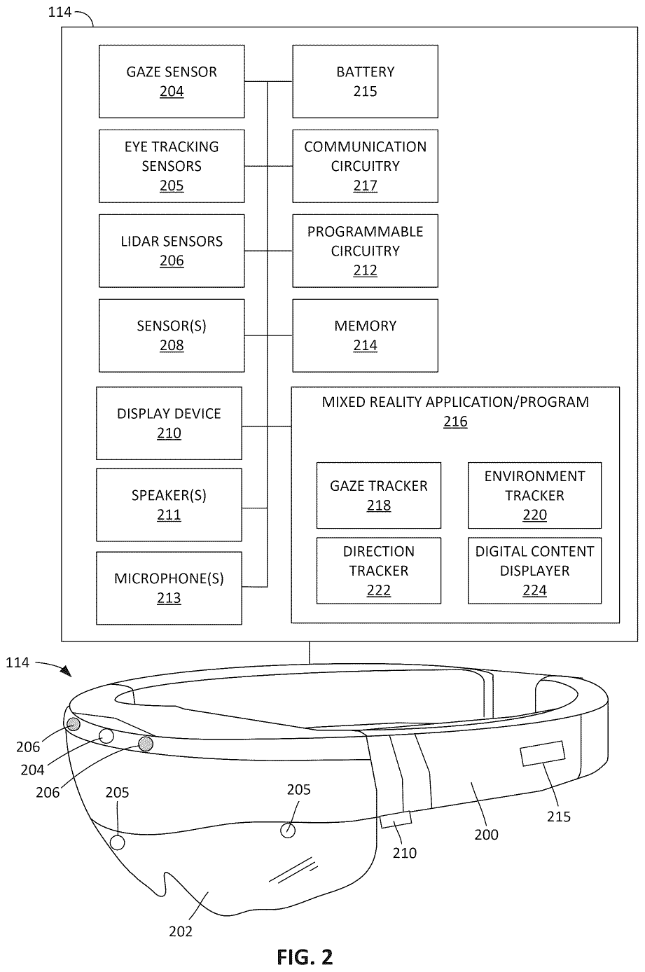

As described above, some people donating blood are uncomfortable (e.g., anxious, fearful, etc.) with having a needle inserted into their arm. To help calm the person 102, the example environment 100 includes an example extended reality device 114 to expose the person 102 to an extended reality environment during the blood collection process. In this example, the extended reality device 114 is a headset 114 (also referred to as a mixed reality headset) that is worn on a head 116 of the person 102 during the blood collection process. In particular, in this example, the headset 114 is worn on the person's head 116 and at least partially covers the person's eyes. The example headset 114 is shown in more detail in FIG. 2. In some examples, the headset 114 is provided to the person 102 before the blood collection procedure starts. For example, the blood donation facility may provide the headset 114 to the person 102 while the person 102 is going through pre-screening and/or filling out paperwork. In another example, the blood donation facility may provide the headset 114 to the person 102 when they are seated, but before the needle 108 is inserted into their arm. The person 102 may put the headset 114 on their head 116 and adjust the headset 114 (e.g., by pulling one or more adjustment straps) to ensure the headset 114 is comfortably situated on the person's head 116. In some examples, the phlebotomist 104 or another person may help the person 102 place the headset 114 on the person's head 116 and/or adjust the headset 114. In some examples, the headset 114 is the Microsoft Hololens headset. In other examples, the headset 114 can be another type of headset, such as for example, the Varjo XR3, Magic Leap 2, or Oculus Quest Pro. In other examples, the extended reality device 114 can be implemented as another head-worn device (e.g., glasses, goggles, a helmet, a hat, etc.) capable of providing a MR experience, such as Google Glass or other smart glasses.

Before the blood collection process begins, the headset 114 is activated and the headset 114 executes an application or program that provides a mixed reality environment to the person 102. The mixed reality environment includes digital content that is displayed or reflected on the glasses (and/or other display) and therefore appears to be located in the surrounding environment. In some examples, the mixed reality environment is an interactive environment, such that the person 102 can interact with the digital content in the mixed reality environment. In some examples, the mixed reality program or application is a game. In other examples, the mixed reality program or application is entirely passive, such that the person 102 only views the mixed reality environment but does not interact with the digital content. The mixed reality environment is continued to be displayed to the person 102 throughout the blood collection process. In some examples, the mixed reality environment is displayed throughout the entire blood collection process, while in other examples the mixed reality environment may only be displayed during a portion of the blood collection process. This mixed reality environment helps direct the person's attention away from the blood collection process and toward the mixed reality experience. As such, the person 102 is calmer and less fearful during the blood collection process. Therefore, persons will be more likely to donate blood, which is beneficial to increase the supply of donated blood.

FIG. 2 illustrates an example physical implementation of the example headset 114. FIG. 2 also shows a block diagram of the headset 114. Certain components are labeled in both the physical implementation and the block diagram. Other components are only labeled in one of the physical implementation or the block diagram. In the illustrated example, the headset 114 includes an example headband 200 to be worn around the head 116 (FIG. 1) of the person 102. In some examples, the headband 200 is adjustable (e.g., able change a diameter or circumference of the headband 200). The headset 114 includes example glasses 202 coupled to and extending from the headband 200. The glasses 202 can also be referred to herein as a visor or face shield, which can be implemented as a piece of plastic or glass that covers a portion of the person's face. The glasses 202 may also be referred to herein as a display because digital content is displayed on the glasses 202, as disclosed in further detail herein. The glasses 202 are positioned to be disposed in front of the eyes of the person 102 when worn on the person's head 116. In some examples, the glasses 202 are in a fixed position relative to the headband 200. In other examples, the glasses 202 can be flipped or rotated relative to the headband 200 (e.g., flipped out of view of the person 102). The glasses 202 are at least partially transparent, translucent, or clear, such that the person 102 can look through the glasses 202 and view real-world surroundings and/or environment through the glasses 202 (e.g., the inside of the room where the blood collection procedure occurs). This enables the person 102 to maintain awareness of their surroundings while engaging with the MR experience, which reduces the risk of disorientation that can sometimes occur in a fully virtual environment. Moreover, having transparent or clear glasses 202 enables a medical professional to monitor one or both of the eye(s) of the person 102 during the blood collection process. Therefore, the medical profession can effectively monitor the person 102 for adverse effects such as a vasovagal event, dizziness, and/or spatial awareness problems. In some examples, the glasses 202 may be tinted.

In the illustrated example, the headset 114 includes an example gaze sensor 204. In some examples, the gaze sensor 204 is mounted on a front of the headband 200 and/or top portion of the glasses 202 and faces outward. The gaze sensor 204 measures or tracks the orientation or direction of the headset 114, which can be used to determine the direction (e.g., vector) the person 102 is looking and/or the focal/gaze point based on the direction or orientation of the headset 114. Additionally or alternatively, the headset 114 can include one or more example eye tracking sensors 205. In some examples, the headset 114 includes two eye tracking sensors 205. The eye tracking sensors 205 can be mounted on the headband 200 and/or on the inside of the glasses 202 and face inward toward the person's eyes. The eye tracking sensors 205 track the person's eyes individually. Thus, the eye tracking sensors 205 determine different measurements of where a person is looking based on each eye. In some examples, the eye tracking sensors 205 track the positions of the person's eyes based on the pupils. Vectors from each eye can be determined based on the eye tracking. This information can be used to determine the intersection of the vectors from each of the person's eyes, which forms the focal/gaze point.

In the illustrated example, the headset 114 includes example light detection and ranging (LiDAR) sensors 206. In some examples, the LiDAR sensors 206 are mounted on the headband 200 or top portion of the glasses 202 and face outward toward the environment in front of the person 102. The LiDAR sensors 206 are used to create a three-dimensional (3D) map and/or mesh of the surrounding surfaces and objects. In some examples, the headset 114 includes one or more example sensor(s) 208, such as for example an accelerometer and/or gyroscope. These sensor(s) 208 can be used to determine the location of the headset 114 and the direction in which the person 102 is looking.

In the illustrated example, the headset 114 includes an example display device 210 to display the digital content. In some examples, the display device 210 is a projector that projects the digital content on the inside of the glasses 202. Additionally or alternatively, the display device 210 can include a display that is mounted on the headband 200 to reflect the digital content off of the inside of the glasses 202. Additionally or alternatively, the display device 210 can include a transparent display that is disposed in front of or behind the glasses 202 or is implemented as the glasses 202. In some examples, the display is an organic light-emitting diode (OLED) display, a flexible OLED (FOLED), and/or other types of displays.

In some examples, the headset 114 includes one or more example speaker(s) 211. In some examples, the headset 114 include one or more microphone(s) 213 (e.g., a microphone array).

In some examples, the headset 114 includes a battery 215 (e.g., one or more batteries, a battery pack), which provides power to the electronics on the headset 114. In some examples, the battery 215 is removably coupled to the headband 200.

In some examples, the headset 114 includes communication circuitry 217, which enables the headset 114 to communicate with another device, such as an electronic device (e.g., a computer, a laptop, a gaming system, a smart phone, etc.). In some examples, the communication circuitry 217 includes wireless circuitry, such as a Bluetooth® transceiver. In other examples, the communication circuitry 217 can include other types of wireless technology.

In the illustrated example, the headset 114 includes example programmable circuitry 212 and example memory 214. In some examples, the programmable circuitry 212 and the memory 214 are mounted in the material of the headband 200. Additionally or alternatively, the programmable circuitry 212 and/or the memory 214 can be coupled to a side of the headband 200. The programmable circuitry 212 is communicative coupled (e.g., via one or more wires or traces) to the gaze sensor 204, the eye tracking sensors 205, the LiDAR sensors 206, the sensor(s) 208, the display 210, the speaker(s) 211, the microphone(s) 213, the communication circuitry 217, and the memory 214.

In the illustrated example, the headset 114 includes an example mixed reality application or program 216 (e.g., software). In some examples, the program 216 is instantiated by the programmable circuitry 212 executing instructions and/or configured to perform one or more operations. The program 216, when executed, creates the mixed reality environment or experience on the headset 114. In some examples, the program 216 is stored in the memory 214. In other examples, the headset 114 can communicate with a remote device (e.g., a computer) that executes the program 216 and communicates the digital content to the headset 114. For example, the wireless circuitry 217 can communicate with a computer in the room or other remote location. The communication circuitry 217 transmits data (e.g., from the gaze sensor 204, the eye tracking sensors 205, the LiDAR sensors 206, the sensor(s) 208) to the computer. The computer executes the program 216, which analyzes the data and determines the digital content to be displayed. In some examples, a plurality of mixed reality programs are stored in the memory and/or on the remote computer. In some examples the person 102 and/or another person can select one of the mixed reality programs to execute on the headset 114 (e.g., by selecting one of the mixed reality programs on a user interface screen).

In some examples, the program 216 of FIG. 2 is instantiated (e.g., creating an instance of, bring into being for any length of time, materialize, implement, etc.) by programmable circuitry such as a Central Processing Unit (CPU) executing first instructions. Additionally or alternatively, the program 216 of FIG. 2 may be instantiated (e.g., creating an instance of, bring into being for any length of time, materialize, implement, etc.) by (i) an Application Specific Integrated Circuit (ASIC) and/or (ii) a Field Programmable Gate Array (FPGA) structured and/or configured in response to execution of second instructions. It should be understood that some or all of the circuitry of FIG. 2 may, thus, be instantiated at the same or different times. Some or all of the circuitry may be instantiated, for example, in one or more threads executing concurrently on hardware and/or in series on hardware. Moreover, in some examples, some or all of the circuitry of FIG. 2 may be implemented by microprocessor circuitry executing instructions and/or FPGA circuitry performing operations to implement one or more virtual machines and/or containers.

In the illustrated example, the program 216 includes an example gaze tracker 218. In some examples, the gaze tracker 218 analyzes the data or measurements from the eye tracking sensors 205 and determines the direction of the person's gaze and/or a focal point (e.g., the intersection of both eye vectors) of the person's eyes. Additionally or alternatively, the gaze tracker 218 can use the data or measurements from the gaze sensor 204. In some examples, the eye tracking sensors 205 are dynamic while the gaze sensor 204 is static. In some examples, the gaze tracker 218 uses the data or measurements from the eye tracking sensors 205 as a primary source to determine the gaze point, and the gaze tracker 218 uses the data or measurements from the gaze sensor 204 as a backup or secondary source if an accurate location cannot be determined from the eye tracking sensors 205.

In the illustrated example, the program 216 includes an example environment tracker 220. The environment tracker 220 analyzes the data from the LiDAR sensors 206 and generates or creates a 3D model, map, and/or mesh of the surrounding environment (also referred to herein as the real-world surroundings). The environment tracker 220 can detect or recognize objects, surfaces, and/or structures in the surrounding environment, such as walls, doors, desks, chairs, etc.

In the illustrated example, the program 216 includes an example direction tracker 222. The direction tracker 222 determines or tracks the direction the headset 114 is facing within the 3D model of the surrounding environment. In some examples, the direction tracker 222 uses input from the gaze sensor 204, the LiDAR sensors 206, and/or the sensor(s) 208.

In the illustrated example, the program 216 includes an example digital content displayer 224. The digital content displayer 224 determines the digital content to be displayed on the glasses 202. The digital content can include any content, such as for example 3D appearing objects, text, windows, colors, shapes, images, videos, etc. The digital content displayer 224 determines what content to display and where to display the content based input from the gaze tracker 218, the environment tracker 220, and/or the direction tracker 222. For example, the digital content displayer 224 may determine to display a digital object (also referred to as a virtual object) on top of a desk in the real work environment. The digital content displayer 224 stores of the location of the digital object relative to the location of the desk in the 3D model. Therefore, when the person 102 is looking in the direction of the desk, the digital content display 224 controls the display device 210 to display the digital object on a portion of the glasses 202 where the desk is viewable, such that the digital object appears to be on top of the desk. The direction tracker 222 continues to track the direction/orientation of the headset as the person looks around, and the digital content displayer 224 adjusts the location of the digital object on the glasses 202. As such, as the person 102 looks around the room, the digital object still appears to stay in the same relative location on the top of the desk.

In some examples, the mixed reality environment created by the program 216 is interactive. For example, the person 104 may provide input that controls and/or effects the digital content in the mixed reality environment. In this example, the person 102 interacts with the mixed reality environment via gaze control, sometimes referred to as eye control or gaze interaction. As used herein, gaze control means causing a computer (programmable circuitry) to take an action based on a direction of a person's gaze, a changing direction of gaze, and/or a length of time a gaze is held. For example, the person 102 may stare or focus at a digital object in the mixed reality environment for a threshold period of time (e.g., 3 seconds) to select the object and/or cause an action. In some examples, this causes a change in one or more of the digital objects in the mixed reality environment based on the gaze direction of the eyes of the person 102 to enable the person 102 to control the one or more of the digital objects. Therefore, the person 102 does not need additional controllers for their hands. This enables the person's hands and arms to remain relaxed and still throughout the blood donation procedure. Therefore, in this example, the person 102 does not interact with the mixed reality environment via a hand-held device.

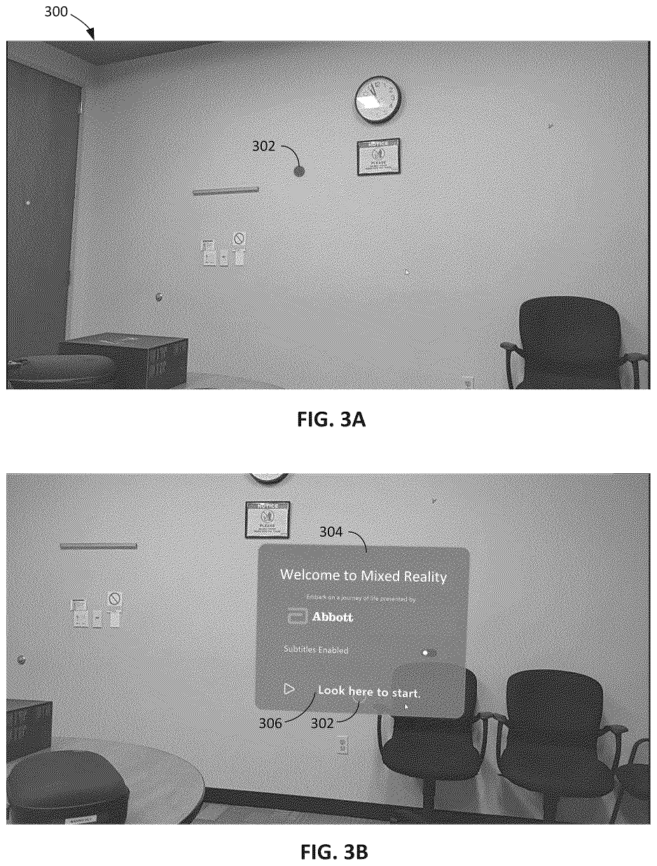

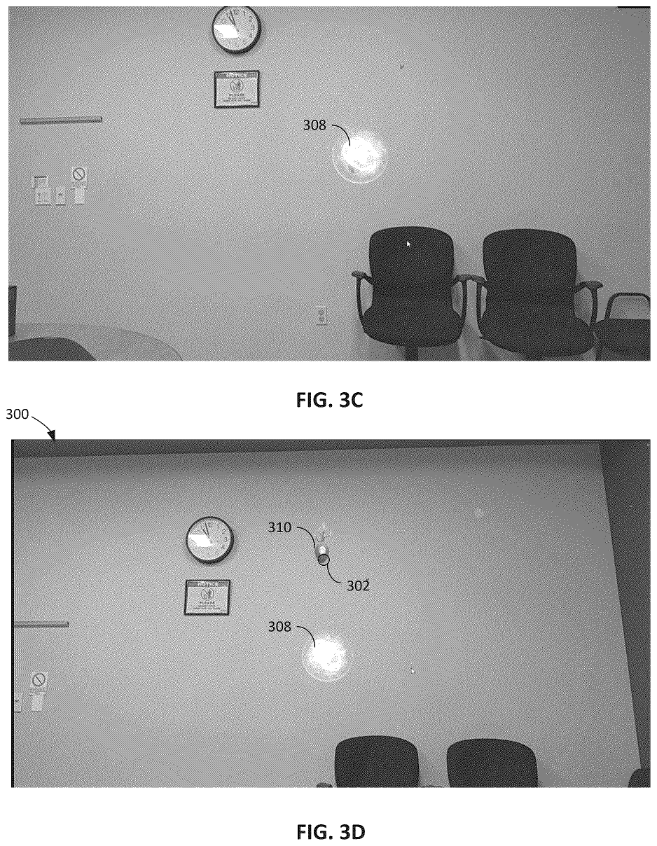

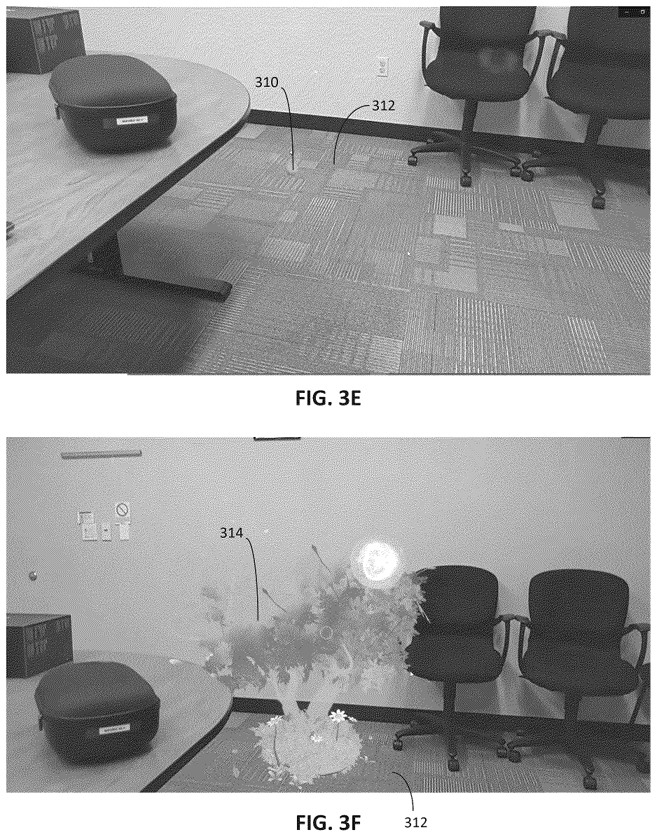

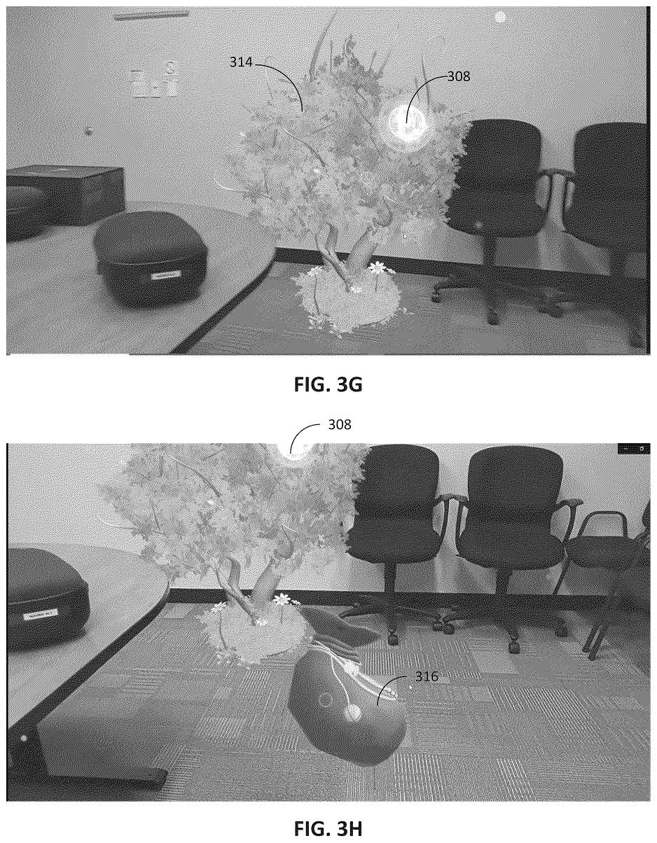

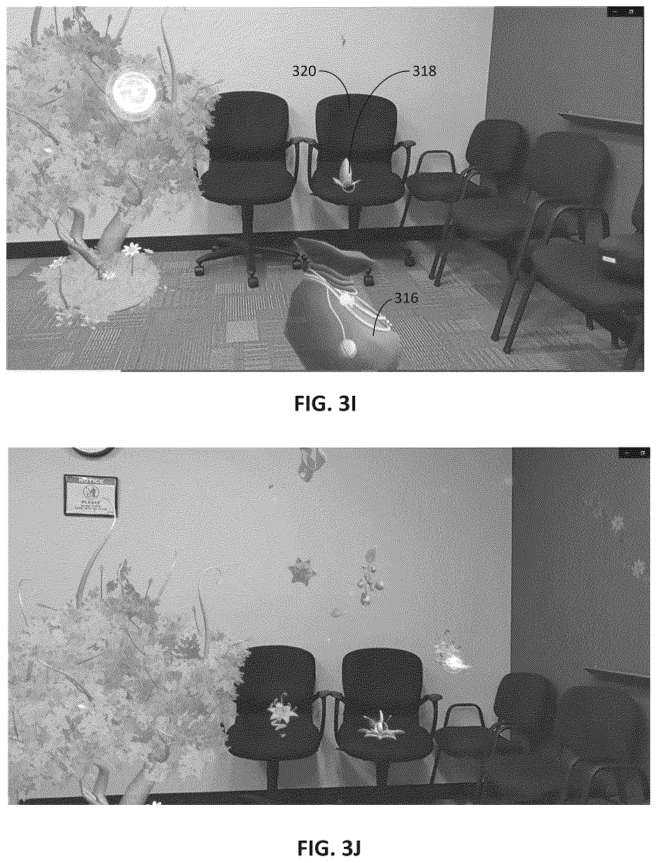

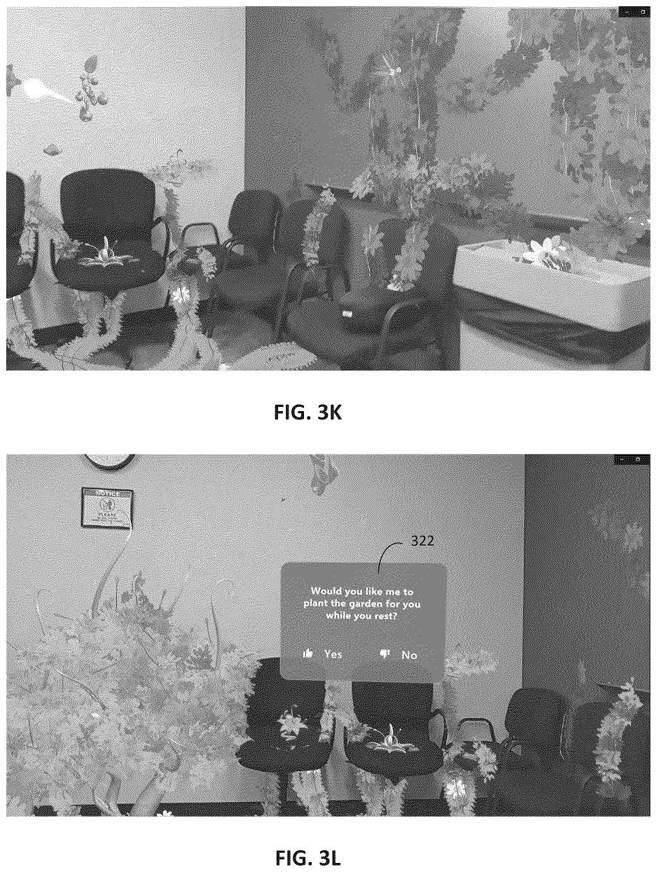

FIGS. 3A-3L show example views of an example mixed reality environment as seen by the person 102 through the headset 114 during an example mixed reality experience provided by the mixed reality program 216. In this example, the program 216 is an interactive program that allows the person 102 to grow one or more plants as part of a garden or forest in the surrounding room where the person 102 is located.

FIG. 3A shows the view from the person 102 looking through the glasses 202 of the headset 114. The area that is viewable through the glasses 114 is referred to herein as the field of view 300. In this example, the person 102 is inside a room with various structures and objects such as a wall, a door, a clock, signs, a chair, a desk, etc. that can be seen in the field of view 300. The person 102 can move his/her head to look around the room and view other areas of the room while the blood collection process occurs. During the mixed reality experience, the environment tracker 220 detects, recognizes, and tracks the various objects, structures, and surfaces in the room and creates a 3D model of the surrounding environment. This 3D model is used by the digital content displayer 224 to position certain digital content at certain locations in the surrounding environment.

In the illustrated example of FIG. 3A, the digital content displayer 224 causes the display device 210 to display a cursor 302 at the location of the person's gaze in the field of view 300. For instance, the gaze tracker 218 determines or tracks the focal point of the person's gaze, and the digital content displayer 224 controls the display device 210 to display the cursor 302 on the glasses 202 at the location of the focal point. In this example, the cursor 302 is a circle. In other examples, the cursor 300 can be implemented as a different shaped object. As the person 102 moves their eyes, the cursor 300 moves to the corresponding location of their focal point. The cursor 300 provides feedback to the person 102 so that the person 102 can tell where they are looking in the mixed reality environment.

As shown in FIG. 3B, the digital content displayer 224 causes the display device 210 to display an introduction window 304, which appears in this example to be displayed over a portion of the wall in the room. The introduction window 304 may include instructions and/or other information. In this example, the introduction window 304 instructs the person 102 to stare at a button 306 (“Look here to start”). The person 102 is supposed to stare at the button for a threshold period of time, such as for example 2 or 3 seconds. Other time periods many be used in other examples for all time thresholds disclosed herein. In this example, when the person 102 looks at the button 206, the cursor 302 is moved over the button 306. If the person 102 keeps the cursor 302 on the button 306 for the threshold period of time, the program 216 continues. In some examples, the cursor 302 shows a time symbol (e.g., a rotating wheel) or other indicator that represents the amount of time the person 102 has looked at the button 306. This provides feedback to the person 102.

In FIG. 3C, the digital content displayer 224 causes the display device 210 to display a virtual avatar 308 that acts as a guide. In this example the avatar 308 is presented as an orb (e.g., a ball of light). In other examples, the avatar 308 can have a different shape. In some examples, the avatar 308 provides audio instructions via the speaker 211. In some examples, the audio instructions explain how the mixed reality program 216 works, how to interact with various digital content, and/or other information associated with the mixed reality experience. The audible instructions are coordinated with the avatar 308 (e.g., with movement or pulsing of the avatar 308). Additionally or alternatively, the digital content displayer 224 can display subtitles with the instructions and/or information. In some examples, the avatar 308 has artificial intelligence (AI) pathing coded to follow the person's vision and avoid obstacles in the surrounding environment. In some examples, the avatar 308 moves around the room and asks the person 102 to look at the avatar 308, which helps introduce the person 102 to the gaze control feature in the mixed reality environment.

In FIG. 3D, digital content displayer 224 causes the display device 210 to display a seed 310. The person 102 is instructed by the avatar 308 to stare at the seed 310, such that the cursor 302 is moved over the seed 310. If the cursor 302 is placed over the seed 310 for a threshold period of time, the seed 310 is selected and thereafter follows the cursor 302. The person 102 can then move the seed 310 (via gaze control) to any location within the field of view 300. When the person 102 stares at a certain location for a threshold period of time, such as for example 2 or 3 seconds, the seed 310 is planted. For example, in FIG. 3E, the person 102 stares at a location on the floor 312 of the room. In some examples, the environment tracker 220 identifies flat zones (e.g., flat surfaces or objects) in the surrounding environment with free space surrounding for the plant to grow (e.g., on the floor, on the seat of the chair, etc.). In some examples, the digital content displayer 244 displays indicators (e.g., green circles) at these locations. Once the location is selected (via gaze control), the environment tracker 220 records this location in the 3D model. The seed 310 is planted and begins to grow from the location on the floor 312. For example, after a few seconds, the seed 310 grows into a plant 314 shown in FIG. 3F. The person 102 can look around the room, but the plant 314 remains in the same relative location sprouting from the floor 312. In some examples, the mixed reality program 216 asks the person 102 to perform other actions using the gaze control feature, such as catching butterflies. This may help the person 102 become comfortable with the gaze control interaction.

In FIGS. 3G and 3H, a bag 316 appears and the avatar 308 instructs the person 102 to pull additional seeds from the bag 316 and continue planting the seeds throughout the surrounding environment. When the person 102 stares at the bag 316, the cursor 302 selects a new seed, which the person 102 can plant in another location in the room. For example, in FIG. 3I, the person 102 places a seed 318 on a chair 320. In some examples, to plant the seed 318, the person 102 needs to stare at the location for a threshold period of time, such as for example 2 or 3 seconds. Once the seed 318 is planted, the seed 318 begins to grow into a plant. The person 102 can continue to pull seeds from the bag 316 and plant the seeds throughout the room. As shown in FIG. 3J, the person 102 has planted seeds in various locations, such as on the chairs, on the wall, etc. The person 102 can watch the seeds grow into plants (e.g., flowers, trees, bushes, etc.) around the room. In some examples, the seeds are programmed to respect gravity and orientation. For example, if a seed is planted on a wall, it grows outward. If a seed is planted on the ceiling, it grows downward.

In some examples, as shown in FIG. 3K, one or more plants may automatically grow around the room. In other words, the digital content 224 displayer may automatically show other plants at other locations growing around the room. For example, as shown in FIG. 3K, a vine is shown as growing across the wall. Soon the room appears as garden, forest, or jungle with various plants.

In some examples, if the person 102 does not wish to interact or is struggling with the gaze control feature, the avatar 308 begins planting seeds for the person 102. For example, in FIG. 3L, the digital content displayer 224 causes the display 210 to display a window 322 asking if the person 102 would like the program 216 to continue automatically planting the garden while the person 102 rests. The person 102 can select (via gaze control) yes or no. In some examples, the mixed reality program 216 has an inactivity timer and prompts the person 102 if they do not interact with the environment within 60 seconds.

This example mixed reality environment of a garden is calming and soothing to the person 102. Helping to grow the garden provides the person 102 with a sense of gratitude and giving back. The mixed reality experience helps draw the person's attention away from the blood collection process. This reduces the person's fear or anxiety with the blood collection process. As such, the person 102 feels calmer and more relaxed during the process. In some examples, the program 216 causes the speaker 211 to play calming music during the MR experience, which helps further calm the person 102. In some examples, the program 216 ends with a positive message about blood donation. Though the example of FIGS. 3A-3L show an interactive program for growing a plant, in other examples any interactive program may be presented that will provide a distraction to the person 102 to reduce anxiety.

While in some examples mixed reality is used to entertain or distract the person 102 during the blood collection process, other medical procedure, and/or other anxiety-inducing situation, in other examples, other extended reality technologies can be used instead. For example, augmented reality or virtual reality can be used. In some such examples, the person 102 is provided an augment reality or virtual reality display device, such as a headset, a tablet, etc. In some examples the display device is worn or held by the person 102. In other examples, the display device can be mounted near the location of the person 102, such that the person 102 does not need to handle the display device. Therefore, an example method disclosed herein includes exposing a person to an extended reality environment during a medical procedure (e.g., donating blood).

While an example manner of implementing the mixed reality program 216 is illustrated in FIG. 2, one or more of the elements, processes, and/or devices illustrated in FIG. 2 may be combined, divided, re-arranged, omitted, eliminated, and/or implemented in any other way. Further, the example gaze tracker 218, the example environment tracker 220, the example direction tracker 222, the example content displayer 224, and/or, more generally, the example mixed reality program 216 of FIG. 2, may be implemented by hardware alone or by hardware in combination with software and/or firmware. Thus, for example, any of the example gaze tracker 218, the example environment tracker 220, the example direction tracker 222, the example content displayer 224, and/or, more generally, the example mixed reality program 216, could be implemented by programmable circuitry in combination with machine readable instructions (e.g., firmware or software), processor circuitry, analog circuit(s), digital circuit(s), logic circuit(s), programmable processor(s), programmable microcontroller(s), graphics processing unit(s) (GPU(s)), digital signal processor(s) (DSP(s)), application specific integrated circuit(s) (ASIC(s)), programmable logic device(s) (PLD(s)), and/or field programmable logic device(s) (FPLD(s)) such as Field Programmable Gate Arrays (FPGAs). Further still, the example mixed reality program 216 of FIG. 2 may include one or more elements, processes, and/or devices in addition to, or instead of, those illustrated in FIG. 2, and/or may include more than one of any or all of the illustrated elements, processes and devices.

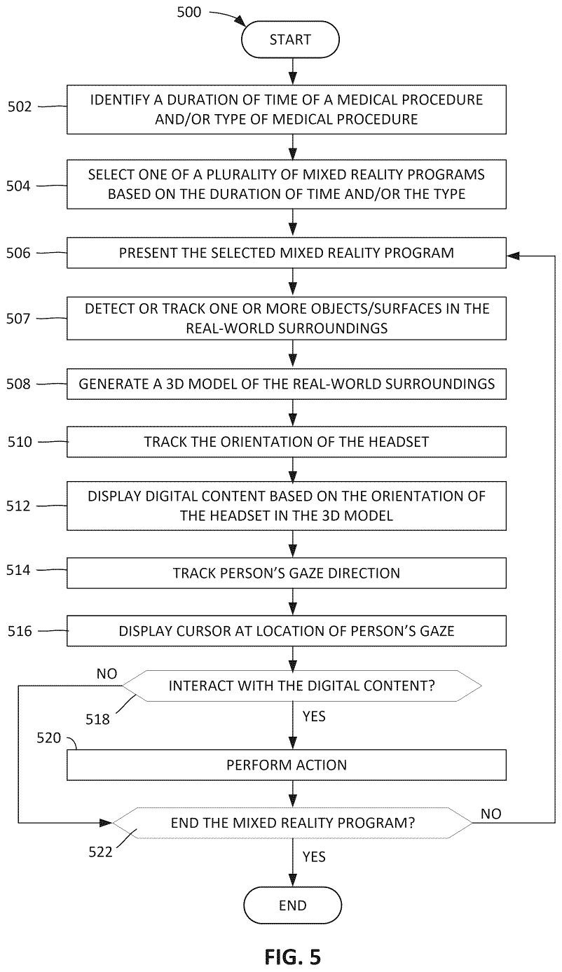

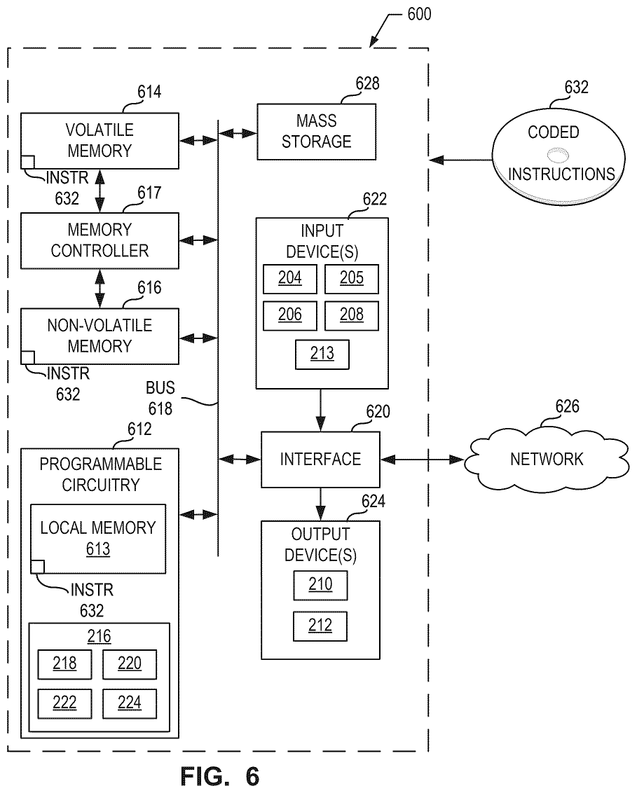

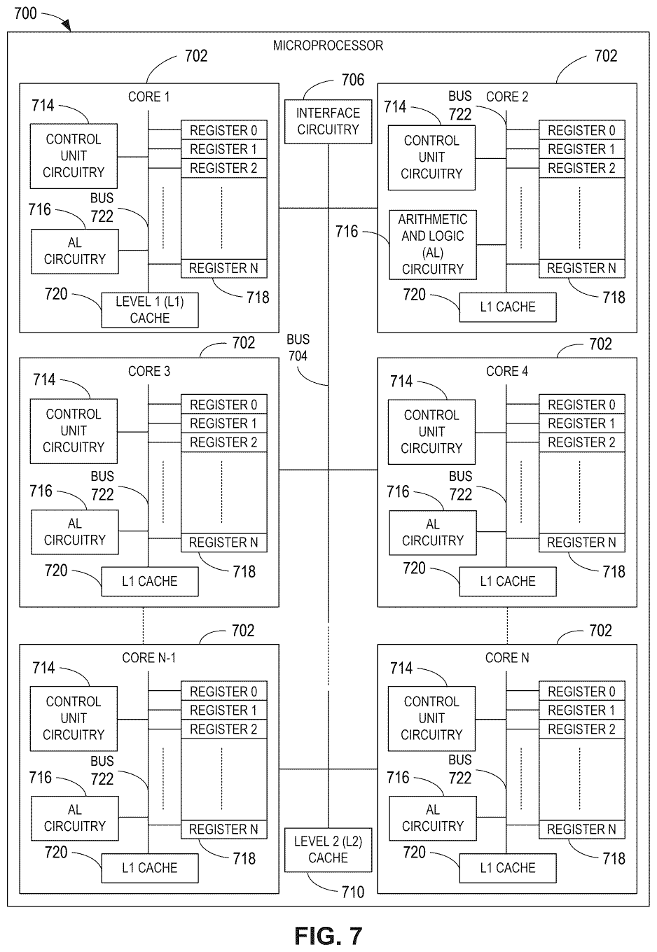

A flowchart representative of example machine readable instructions, which may be executed by programmable circuitry to implement and/or instantiate the mixed reality program 216 of FIG. 2 and/or representative of example operations which may be performed by programmable circuitry to implement and/or instantiate the mixed reality program 216 of FIG. 2, is shown in FIG. 5. The machine readable instructions may be one or more executable programs or portion(s) of one or more executable programs for execution by programmable circuitry, such as the programmable circuitry 612 shown in the example processor platform 600 discussed below in connection with FIG. 6 and/or may be one or more function(s) or portion(s) of functions to be performed by the example programmable circuitry (e.g., an FPGA) discussed below in connection with FIGS. 7 and/or 8. In some examples, the machine readable instructions cause an operation, a task, etc., to be carried out and/or performed in an automated manner in the real world. As used herein, “automated” means without human involvement.

The program may be embodied in instructions (e.g., software and/or firmware) stored on one or more non-transitory computer readable and/or machine readable storage medium such as a cache memory, a magnetic-storage device or disk (e.g., a floppy disk, a Hard Disk Drive (HDD), etc.), an optical-storage device or disk (e.g., a Blu-ray disk, a Compact Disk (CD), a Digital Versatile Disk (DVD), etc.), a Redundant Array of Independent Disks (RAID), a register, ROM, a solid-state drive (SSD), SSD memory, non-volatile memory (e.g., electrically erasable programmable read-only memory (EEPROM), flash memory, etc.), volatile memory (e.g., Random Access Memory (RAM) of any type, etc.), and/or any other storage device or storage disk. The instructions of the non-transitory computer readable and/or machine readable medium may program and/or be executed by programmable circuitry located in one or more hardware devices, but the entire program and/or parts thereof could alternatively be executed and/or instantiated by one or more hardware devices other than the programmable circuitry and/or embodied in dedicated hardware. The machine readable instructions may be distributed across multiple hardware devices and/or executed by two or more hardware devices (e.g., a server and a client hardware device). For example, the client hardware device may be implemented by an endpoint client hardware device (e.g., a hardware device associated with a human and/or machine user) or an intermediate client hardware device gateway (e.g., a radio access network (RAN)) that may facilitate communication between a server and an endpoint client hardware device. Similarly, the non-transitory computer readable storage medium may include one or more mediums. Further, although the example program is described with reference to the flowchart illustrated in FIG. 5, many other methods of implementing the example mixed reality program 216 may alternatively be used. For example, the order of execution of the blocks of the flowchart may be changed, and/or some of the blocks of the flowchart described may be changed, eliminated, or combined. Additionally or alternatively, any or all of the blocks may be implemented by one or more hardware circuits (e.g., processor circuitry, discrete and/or integrated analog and/or digital circuitry, an FPGA, an ASIC, a comparator, an operational-amplifier (op-amp), a logic circuit, etc.) structured to perform the corresponding operation without executing software or firmware. The programmable circuitry may be distributed in different network locations and/or local to one or more hardware devices (e.g., a single-core processor (e.g., a single core central processor unit (CPU)), a multi-core processor (e.g., a multi-core CPU, an XPU, etc.). For example, the programmable circuitry may be a CPU and/or an FPGA located in the same package (e.g., the same integrated circuit (IC) package or in two or more separate housings), one or more processors in a single machine, multiple processors distributed across multiple servers of a server rack, multiple processors distributed across one or more server racks, etc. and/or any combination(s) thereof.

The machine readable instructions described herein may be stored in one or more of a compressed format, an encrypted format, a fragmented format, a compiled format, an executable format, a packaged format, etc. Machine readable instructions as described herein may be stored as data (e.g., computer-readable data, machine-readable data, one or more bits (e.g., one or more computer-readable bits, one or more machine-readable bits, etc.), a bitstream (e.g., a computer-readable bitstream, a machine-readable bitstream, etc.), etc.) or a data structure (e.g., as portion(s) of instructions, code, representations of code, etc.) that may be utilized to create, manufacture, and/or produce machine executable instructions. For example, the machine readable instructions may be fragmented and stored on one or more storage devices, disks, and/or computing devices (e.g., servers) located at the same or different locations of a network or collection of networks (e.g., in the cloud, in edge devices, etc.). The machine readable instructions may require one or more of installation, modification, adaptation, updating, combining, supplementing, configuring, decryption, decompression, unpacking, distribution, reassignment, compilation, etc., in order to make them directly readable, interpretable, and/or executable by a computing device and/or other machine. For example, the machine readable instructions may be stored in multiple parts, which are individually compressed, encrypted, and/or stored on separate computing devices, wherein the parts when decrypted, decompressed, and/or combined form a set computer-executable and/or of machine executable instructions that implement one or more functions and/or operations that may together form a program such as that described herein.

In another example, the machine readable instructions may be stored in a state in which they may be read by programmable circuitry, but require addition of a library (e.g., a dynamic link library (DLL)), a software development kit (SDK), an application programming interface (API), etc., in order to execute the machine readable instructions on a particular computing device or other device. In another example, the machine readable instructions may need to be configured (e.g., settings stored, data input, network addresses recorded, etc.) before the machine readable instructions and/or the corresponding program(s) can be executed in whole or in part. Thus, machine readable and/or computer readable media, as used herein, may include instructions and/or program(s) regardless of the particular format or state of the machine readable instructions and/or program(s).

The machine readable instructions described herein can be represented by any past, present, or future instruction language, scripting language, programming language, etc. For example, the machine readable instructions may be represented using any of the following languages: C, C++, Java, C#, Perl, Python, JavaScript, HyperText Markup Language (HTML), Structured Query Language (SQL), Swift, etc.

As mentioned above, the example operations of FIG. 5 may be implemented using executable instructions (e.g., computer readable and/or machine readable instructions) stored on one or more non-transitory computer readable and/or machine readable media. As used herein, the terms non-transitory computer readable medium, non-transitory computer readable storage medium, non-transitory machine readable medium, and/or non-transitory machine readable storage medium are expressly defined to include any type of computer readable storage device and/or storage disk and to exclude propagating signals and to exclude transmission media. Examples of such non-transitory computer readable medium, non-transitory computer readable storage medium, non-transitory machine readable medium, and/or non-transitory machine readable storage medium include optical storage devices, magnetic storage devices, an HDD, a flash memory, a read-only memory (ROM), a CD, a DVD, a cache, a RAM of any type, a register, and/or any other storage device or storage disk in which information is stored for any duration (e.g., for extended time periods, permanently, for brief instances, for temporarily buffering, and/or for caching of the information). As used herein, the terms “non-transitory computer readable storage device” and “non-transitory machine readable storage device” are defined to include any physical (mechanical and/or electrical) hardware to retain information for a time period, but to exclude propagating signals and to exclude transmission media. Examples of non-transitory computer readable storage devices and machine readable storage devices include random access memory of any type, read only memory of any type, solid state memory, flash memory, optical discs, magnetic disks, disk drives, and/or redundant array of independent disks (RAID) systems. As used herein, the term “device” refers to physical structure such as mechanical and/or electrical equipment, hardware, and/or circuitry that may or may not be configured by computer readable instructions, machine readable instructions, etc., and/or manufactured to execute computer readable instructions, machine readable instructions, etc.

“Including” and “comprising” (and all forms and tenses thereof) are used herein to be open ended terms. Thus, whenever a claim employs any form of “include” or “comprise” (e.g., comprises, includes, comprising, including, having, etc.) as a preamble or within a claim recitation of any kind, it is to be understood that additional elements, terms, etc., may be present without falling outside the scope of the corresponding claim or recitation. As used herein, when the phrase “at least” is used as the transition term in, for example, a preamble of a claim, it is open-ended in the same manner as the term “comprising” and “including” are open ended. The term “and/or” when used, for example, in a form such as A, B, and/or C refers to any combination or subset of A, B, C such as (1) A alone, (2) B alone, (3) C alone, (4) A with B, (5) A with C, (6) B with C, or (7) A with B and with C. As used herein in the context of describing structures, components, items, objects and/or things, the phrase “at least one of A and B” is intended to refer to implementations including any of (1) at least one A, (2) at least one B, or (3) at least one A and at least one B. Similarly, as used herein in the context of describing structures, components, items, objects and/or things, the phrase “at least one of A or B” is intended to refer to implementations including any of (1) at least one A, (2) at least one B, or (3) at least one A and at least one B. As used herein in the context of describing the performance or execution of processes, instructions, actions, activities and/or steps, the phrase “at least one of A and B” is intended to refer to implementations including any of (1) at least one A, (2) at least one B, or (3) at least one A and at least one B. Similarly, as used herein in the context of describing the performance or execution of processes, instructions, actions, activities and/or steps, the phrase “at least one of A or B” is intended to refer to implementations including any of (1) at least one A, (2) at least one B, or (3) at least one A and at least one B.

As used herein, singular references (e.g., “a”, “an”, “first”, “second”, etc.) do not exclude a plurality. The term “a” or “an” object, as used herein, refers to one or more of that object. The terms “a” (or “an”), “one or more”, and “at least one” are used interchangeably herein. Furthermore, although individually listed, a plurality of means, elements, or actions may be implemented by, e.g., the same entity or object. Additionally, although individual features may be included in different examples or claims, these may possibly be combined, and the inclusion in different examples or claims does not imply that a combination of features is not feasible and/or advantageous.

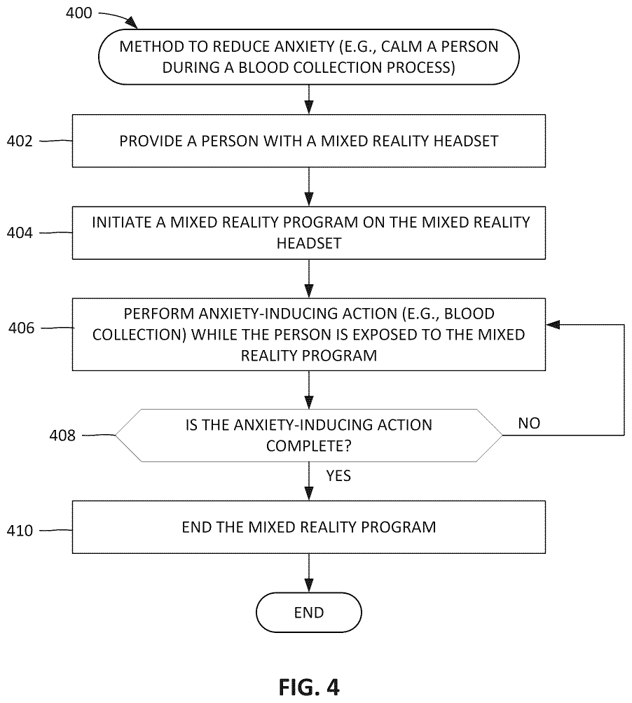

FIG. 4 is a flowchart of an example method 400 that can be implemented to reduce anxiety in a person during an anxiety-inducing process or situation. The example method 400 is describe in connection with a blood collection process. However, the method 400 can be similarly performed in connection with any other type of process or situation.

At block 402, the method 400 includes providing a person with a mixed reality headset. For example, as disclosed above in connection with FIG. 1, a person (e.g., an employee, a nurse, a phlebotomist, etc.) at the blood donation facility can provide the headset 114 to the person 102. In some examples, headset 114 is provided to the person 102 before the person 102 enters the room where the blood collection procedure occurs. For example, the headset 114 may be provided to the person 102 in a waiting room or pre-screening room. In other examples, the headset 114 can be provided to the person 102 in the same room where the blood collection procedure occurs. In some examples, the headset 114 is provided to the person 102 before the person 102 sits or lays in the chair 106. In other examples, the headset 114 is provided to the person 102 after the person 102 is sitting or laying in the chair 106, but before the blood collection procedure occurs. The method 400 includes placing the headset 114 on the person's head 116. In some examples, the person 102 places the headset 114 on their head. Additionally or alternatively, another person (e.g., the phlebotomist 104) may help place the headset 114 on the person's head. In some examples, the person 102 and/or another person can adjust the headset 114 (e.g., by adjusting one or more tension straps) to ensure the headset 114 is comfortable on the person's head 116 and the glasses 202 are positioned in front of the person's eyes.

At block 404, the method 400 includes initiating a mixed reality program on the mixed reality headset. For example, the person 102 and/or another person may activate the headset 114 (e.g., turn the headset 114 on). In some examples, the mixed reality program 216 starts automatically when the headset 114 is activated. Alternatively, the headset 114 may be powered on, and the person 102 and/or another person can activate the mixed reality program 216 by interacting with a user interface on the headset 114 and/or another device (e.g., a computer) that controls the headset 114. Once the mixed reality program 216 is initiated, digital content is displayed on the glasses 202 that creates a mixed reality environment for the person 102. An example of the mixed reality environment is shown in FIGS. 3A-3L. In some examples, such as shown in FIGS. 3A-3L, the mixed reality environment is directed to nature (e.g., including images of plants), which is generally calming. In some examples, the mixed reality environment is interactive, such that the person 102 can interact (e.g., via gaze control) with one or more of the digital objects.

At block 406, the method 400 includes performing an anxiety-inducing action (e.g., event, process, procedure, etc.) such as, for example, a blood collection process, while the person is exposed to the mixed reality program. For example, the phlebotomist 104 performs the blood collection process by inserting the needle 108 into the person's arm. The blood is collected in the bag 112. While the blood is being collected, the phlebotomist 104 monitors the person 102 (e.g., monitors the person's vital signs). The mixed reality program 216 is initiated prior to starting the blood collection process. Therefore, during this process, the person 102 continues to be exposed to the mixed reality program 216 via the headset 114. The mixed reality program 216 entertains the person 102 and/or directs the person's attention away from the blood collection process. As such, the person 102 is calmer and more relaxed during the blood collection process.

At block 408, the method 400 includes determining whether the anxiety-inducing action (e.g., blood collection process) is complete. If the anxiety-inducing action is ongoing (e.g., blood is still being collected), the person 102 continues to use the headset 114 to experience the mixed reality environment. In some examples for blood collection, the blood collection process occurs until a threshold amount of blood has been collected, such as for example 0.5L. In other examples, the threshold amount of blood may be higher (e.g., 1L) or lower (e.g., 0.25L). In some examples, the blood collection process occurs for about 8-10 minutes. In other examples, the blood collection process can last a longer or shorter amount of time. In other examples, the blood collection process may be stopped for another reason, such as if the person's vital signs indicate the person 102 is not feeling well.

When the anxiety-inducing action is complete (e.g., when phlebotomist 104 removes the needle 108 and/or otherwise stops the blood collection process), the process is over. At block 410, the examples method 400 includes ending the mixed reality program 216. For example, the program 216 may be deactivated by exiting the program 216 on the headset 114, turning off the headset 114, and/or removing the headset 114 from the person's head 116.

FIG. 5 is a flowchart representative of example machine readable instructions and/or example operations 500 that may be executed, instantiated, and/or performed by programmable circuitry to provide a person with a mixed reality experience. The instruction 500 may be stored on the memory 214 and executed by the programmable circuitry 212 to implement a mixed reality program, such as the mixed reality program 216.

In some examples, multiple mixed reality programs may be stored on the headset 114 and/or the remote computer operating the programs for viewing on the headset 114. Therefore, in some examples, the person 102 and/or another person can select one of the mixed reality programs to view, such as by interacting with a user interface screen. In some examples, the instructions 500 may select one of the mixed reality programs to execute based on a duration of time of a medical procedure, such as the blood collection process, and/or the type of medical procedure. At block 502, the programmable circuitry 212 identities a duration of time of a medical procedure and/or a type of medical procedure. In some examples, this information is input to the headset 114 by the person 102 and/or another person (e.g., the phlebotomist 104). At block 504, the programmable circuitry 212 selects one of the mixed reality programs, such as the program 216, based on the duration of time and/or the type of medical procedure. For example, the programmable circuitry 212 may select a mixed reality program that has a length that is greater than the anticipated length of the procedure to ensure the mixed reality experience does not end before the medical procedure is over. Additionally or alternatively, the programmable circuitry 212 may select a mixed reality program based on the level of anxiety that may be induced during the procedure. For example, if the procedure is a type that is more serious, the programmable circuitry 212 may select a mixed reality program that is more engaging and stimulating to help draw the person's attention away from the procedure. At block 506, the programmable circuitry 212 executes and/or otherwise presents the selected mixed reality program, such as the program 216. In other examples, there may be only one program on the headset 114. As such, there may not be any selection of a program.

At block 507, the environment tracker 220 detects, recognizes, and tracks objects, surfaces, and/or structures in the real-world surroundings based on measurements from the LiDAR sensors 206.

At block 508, the environment tracker 220 generates a 3D model of the real-world surroundings based on the detected/tracked objects, surfaces, and/or structures. In particular, the environment tracker 220 analyzes the data from the LiDAR sensors 206 and generates a 3D model, map, and/or mesh of the real-world surroundings.

At block 510, the direction tracker 222 tracks or determines the orientation or direction of the headset 114 in the 3D model of the surrounding environment. For example, the direction tracker 222 may use input from the gaze sensor 204, the LiDAR sensors 206, and/or the sensor(s) 208 (e.g., an accelerometer, a gyroscope).

At block 512, the digital content displayer 224 causes the display device 210 to display digital content (e.g., text, images, 3D appearing objects, etc.) in accordance with the program 216 on the glasses 202 based on the orientation or direction of the headset 114 in the 3D model. As such, the digital content appears to the person 102 to be located in the real-world environment, thereby providing a mixed reality experience. As the person 102 moves their head, the display device 210 can change the location of the digital content on the glasses 202 so that the digital content appears to remain in the same relative location in the real-world environment. In some examples, as disclosed in connection with the program in FIGS. 3A-3L, the digital content displayer 224 presents the avatar 308 on the glasses 202 (a display) of the headset 114. The avatar 308 appears within the real-world surroundings, as shown in FIG. 3C. The avatar 308 distracts the person 102 and helps to reduce anxiety in the person 102. In some examples, the digital content display 224 presents the avatar 308 in different positions on the glasses 202 (the display) such that the avatar 308 appears to move around the real-world surroundings to train the person 102 with eye gaze control. In some examples, the digital content displayer 224 present an animation of a moving object (e.g., the avatar 308) on the glasses 202 (the display) and appearing in the real-world surroundings and prompts the person 102 (e.g., via visual instructions on the glasses 202, via audio instructions through the speaker(s) 211) to follow the moving object with eye gaze. In some examples, the digital content displayer 224 presents one or more other digital objects or images, such as images of seeds, plants, etc.

In some examples, the mixed reality program 216 is interactive, such that the person 102 can interact with the digital content. In some examples, the interaction is controlled via gaze control. At block 514, the gaze tracker 218 tracks the direction of the person's gaze based on input from the eye tracking sensors 205 and/or gaze sensor 204. At block 516, the digital content displayer 224 causes the display device 210 to display the cursor 302 at the location of the person's gaze. This provides feedback to the person 102. The person can move the cursor 302 (by changing the direction of their gaze) to certain areas and interact with the digital content.

At block 518, the digital content displayer 224 determines if the person 102 has interacted with the digital content, such as keeping the cursor 302 on a digital object for a threshold period of time. For example, the digital content displayer 224 may determine an amount of time the person 102 holds the focal point (e.g., holds their gaze direction on a certain portion of the glasses 202 containing a digital object) and compares the amount of time to a threshold period of time. If the amount of time satisfies the threshold period of time, at block 520, the digital content displayer 224 determines an action to be performed and causes the display device 210 to perform the action (e.g., selecting a seed, moving the seed, etc.). Therefore, the digital content displayer 224 causes a change in one or more of the digital/virtual objects in the mixed reality environment based on the gaze direction or focal point of the eyes of the person 102 to enable the person 102 to control the one or more of the virtual objects. In other words, presentation of mixed reality program 216 is changed based on the gaze.

As an example operation, the digital content displayer 224 can present a first digital image on the glasses 202 (the display). For example, the first digital image may be an image of a seed, such as the seed 310 shown in FIG. 3D. The position of the first digital image (e.g., the seed 310) is based on the 3D model such that the first digital image (e.g., the seed 310) appears on one or more of the objects or surfaces in the real-world surroundings. For example, as shown in FIG. 3E, the seed 310 appears on the floor 312 in the room. In some examples, the digital content displayer 224 presents the avatar 308 on a first portion of the glasses 202 (the display) and presents the first digital image (e.g., the seed 310) on a second portion of the glasses 202 (the display), the second portion different than the first portion. For example, as shown in FIG. 3D, the avatar 308 is displayed in a different location than the seed 310.

In some examples, the gaze tracker 218 determines or tracks a focal point of the person 102 based on a gaze direction of the eye(s) of the person 102. The digital content displayer 224 determines an amount of time the person 102 holds the focal point and compares the amount of time to a threshold period of time. In response to the amount of time satisfying the threshold, the digital content displayer 224 presents a second digital image on the glasses 202 (the display) in place of the first digital image. The position of the second digital image on the glasses 202 (the display) is based on the 3D model such that the second digital image appears on one or more of the objects or surfaces in the real-world surroundings. For example, as disclosed in connection with FIGS. 3E and 3F, if the person 102 stares or focuses on a spot on the floor 312 for a threshold time (e.g., three seconds), the plant 314 (a second digital image) is presented in place of the seed 310 (the first digital image). In some examples, the environment tracker 220 identifies flatness of the objects and surfaces in the 3D model of the real-world surroundings, and the digital content displayer 224 identifies the position of the second digital image (e.g., the plant 314) based on the flatness.

In some examples, the digital content displayer 224 animates a transformation of the first digital image to the second digital image. For example, as shown in FIGS. 3D-3K, the seeds are animated as transforming into plants that are growing around the room. In some examples, the digital content displayer 224 causes the display device 210 to present a transformation of a digital object based on the amount of time the person holds their focal point satisfying a threshold time. For example, if the person 102 is controlling the seed 310 and stares at a spot on the floor 312 for a certain period of time, the seed 310 is planted and transforms into the plant 314.

In some examples, the digital content displayer 224 assesses an activity level of the person 102 based on the eye gaze (as tracked by the gaze tracker 218). For example, the digital content displayer 224 may analyze the amount of eye movement and determine if the amount of eye movement is relatively low (e.g., indicative of boredom or distraction) or relatively high (e.g., indicative of active engagement). In some examples, the digital content displayer 224 compares the activity level to a threshold level of activity. In some examples, the threshold level of activity is based on time. When the activity level does not satisfy the threshold level of activity, the digital content displayer 224 automatically presents a sequence of additional digital images on the glasses 202 (the display). For example, as shown in FIGS. 3K and 3L, the digital content displayer 224 may present additional plants on the walls, ceiling, etc. that continue to grow around the room. Therefore, even if the person 102 is not actively planting new seeds, the mixed reality environment continues to provide additional content to entertain and/or distract the person 102.

At block 522, the machine readable instructions and/or the operations 500 determine if the mixed reality program 216 has ended. If not, control proceeds back to block 504 and the mixed reality program 216 continues to track the orientation of the headset and display digital content. Otherwise, the example process ends.

FIG. 6 is a block diagram of an example processor platform 600 structured to execute and/or instantiate the machine readable instructions and/or the operations of FIG. 5 to implement the mixed reality program 216 of FIG. 2. The processor platform 600 can be, for example, a server, a personal computer, a workstation, a self-learning machine (e.g., a neural network), a mobile device (e.g., a cell phone, a smart phone, a tablet such as an iPad™), a personal digital assistant (PDA), an Internet appliance, a DVD player, a CD player, a digital video recorder, a Blu-ray player, a gaming console, a personal video recorder, a set top box, a headset (e.g., a mixed reality (MR) headset, an augmented reality (AR) headset, a virtual reality (VR) headset, etc.) or other wearable device, or any other type of computing device.

The processor platform 600 of the illustrated example includes programmable circuitry 612. The programmable circuitry 612 of the illustrated example is hardware. For example, the programmable circuitry 612 can be implemented by one or more integrated circuits, logic circuits, FPGAs, microprocessors, CPUs, GPUs, DSPs, and/or microcontrollers from any desired family or manufacturer. The programmable circuitry 612 may be implemented by one or more semiconductor based (e.g., silicon based) devices. The programmable circuitry 612 may correspond to the programmable circuitry 212. In this example, the programmable circuitry 612 implements the mixed reality program 216 including the gaze tracker 218, the environment tracker 220, the direction tracker 222, and the digital content displayer 224.

The programmable circuitry 612 of the illustrated example includes a local memory 613 (e.g., a cache, registers, etc.). The programmable circuitry 612 of the illustrated example is in communication with a main memory including a volatile memory 614 and a non-volatile memory 616 by a bus 618. The volatile memory 614 may be implemented by Synchronous Dynamic Random Access Memory (SDRAM), Dynamic Random Access Memory (DRAM), RAMBUS® Dynamic Random Access Memory (RDRAM®), and/or any other type of RAM device. The non-volatile memory 616 may be implemented by flash memory and/or any other desired type of memory device. Access to the main memory 614, is controlled by a memory controller 617. In some examples, the memory controller 617 may be implemented by one or more integrated circuits, logic circuits, microcontrollers from any desired family or manufacturer, or any other type of circuitry to manage the flow of data going to and from the main memory 614, 616.

The programmable platform 600 of the illustrated example also includes interface circuitry 620. The interface circuitry 620 may be implemented by hardware in accordance with any type of interface standard, such as an Ethernet interface, a universal serial bus (USB) interface, a Bluetooth® interface, a near field communication (NFC) interface, a Peripheral Component Interconnect (PCI) interface, and/or a Peripheral Component Interconnect Express (PCIe) interface.

In the illustrated example, one or more input devices 622 are connected to the interface circuitry 620. The input device(s) 622 permit(s) a user (e.g., a human user, a machine user, etc.) and/or device to enter data and/or commands into the programmable circuitry 612. The input device(s) 622 can include the gaze sensor 204, the eye tracking sensors 205, the LiDAR sensors 206, the sensor(s) 208, and/or the microphone(s) 213. Additionally or alternatively, the input device(s) 622 can be implemented by, for example, an audio sensor, a microphone, a camera, a keyboard, a button, a mouse, a touchscreen, a track-pad, a trackball, an isopoint device, and/or a voice recognition system.

One or more output devices 624 are also connected to the interface circuitry 620 of the illustrated example. The output device(s) 424 can be implemented, for example, by display devices (e.g., a light emitting diode (LED), an organic light emitting diode (OLED), a liquid crystal display (LCD), a cathode ray tube (CRT) display, an in-place switching (IPS) display, a touchscreen, etc.) such as the display device 210, a tactile output device, a printer, and/or speaker such as the speaker(s) 211. The interface circuitry 620 of the illustrated example, thus, typically includes a graphics driver card, a graphics driver chip, and/or graphics processor circuitry such as a GPU.

The interface circuitry 620 of the illustrated example also includes a communication device such as a transmitter, a receiver, a transceiver, a modem, a residential gateway, a wireless access point, and/or a network interface to facilitate exchange of data with external machines (e.g., computing devices of any kind) by a network 626. The communication can be by, for example, an Ethernet connection, a digital subscriber line (DSL) connection, a telephone line connection, a coaxial cable system, a satellite system, a beyond-line-of-site wireless system, a line-of-site wireless system, a cellular telephone system, an optical connection, etc.

The programmable platform 600 of the illustrated example also includes one or more mass storage discs or devices 628 to store firmware, software, and/or data. Examples of such mass storage discs or devices 628 include magnetic storage devices (e.g., floppy disk, drives, HDDs, etc.), optical storage devices(e.g., Blu-ray disks, CDs, DVDs, etc.), RAID systems, and/or solid state storage discs or devices such as flash memory devices and/or SSDs.

The machine readable instructions 632, which may be implemented by the machine readable instructions of FIG. 5, may be stored in the mass storage device 628, in the volatile memory 614, in the non-volatile memory 616, and/or at least one non-transitory computer readable storage medium such as a CD or DVD that may be removable.

FIG. 7 is a block diagram of an example implementation of the programmable circuitry 612 of FIG. 6. In this example, the programmable circuitry 612 of FIG. 6 is implemented by a microprocessor 700. For example, the microprocessor 700 may be a general-purpose microprocessor (e.g., general purpose microprocessor circuitry). The microprocessor 700 executes some or all of the machine readable instructions of the flowchart of FIG. 5 to effectively instantiate the circuitry of FIG. 2 as logic circuits to perform the operations corresponding to those machine readable instructions. In some such examples, the circuitry of FIG. 2 is instantiated by the hardware circuits of the microprocessor 700 in combination with the machine-readable instructions. For example, the microprocessor 700 may be implemented by multi-core hardware circuitry such as a CPU, a DSP, a GPU, an XPU, etc. Although it may include any number of example cores 702 (e.g., 1 core), the microprocessor 700 of this example is a multi-core semiconductor device including N cores. The cores 702 of the microprocessor 700 may operate independently or may cooperate to execute machine readable instructions. For example, machine code corresponding to a firmware program, an embedded software program, or a software program may be executed by one of the cores 702 or may be executed by multiple ones of the cores 702 at the same or different times. In some examples, the machine code corresponding to the firmware program, the embedded software program, or the software program is split into threads and executed in parallel by two or more of the cores 702. The software program may correspond to a portion or all of the machine readable instructions and/or operations represented by the flowchart of FIG. 5.

The cores 702 may communicate by a first example bus 704. In some examples, the first bus 704 may be implemented by a communication bus to effectuate communication associated with one(s) of the cores 702. For example, the first bus 704 may be implemented by at least one of an Inter-Integrated Circuit (I2C) bus, a Serial Peripheral Interface (SPI) bus, a PCI bus, or a PCIe bus. Additionally or alternatively, the first bus 704 may be implemented by any other type of computing or electrical bus. The cores 702 may obtain data, instructions, and/or signals from one or more external devices by example interface circuitry 706. The cores 702 may output data, instructions, and/or signals to the one or more external devices by the interface circuitry 706. Although the cores 702 of this example include example local memory 720 (e.g., Level 1 (L1) cache that may be split into an L1 data cache and an L1 instruction cache), the microprocessor 700 also includes example shared memory 710 that may be shared by the cores (e.g., Level 2 (L2 cache)) for high-speed access to data and/or instructions. Data and/or instructions may be transferred (e.g., shared) by writing to and/or reading from the shared memory 710. The local memory 720 of each of the cores 702 and the shared memory 710 may be part of a hierarchy of storage devices including multiple levels of cache memory and the main memory (e.g., the main memory 614, 616 of FIG. 6). Typically, higher levels of memory in the hierarchy exhibit lower access time and have smaller storage capacity than lower levels of memory. Changes in the various levels of the cache hierarchy are managed (e.g., coordinated) by a cache coherency policy.

Each core 702 may be referred to as a CPU, DSP, GPU, etc., or any other type of hardware circuitry. Each core 702 includes control unit circuitry 714, arithmetic and logic (AL) circuitry (sometimes referred to as an ALU) 716, a plurality of registers 718, the local memory 720, and a second example bus 722. Other structures may be present. For example, each core 702 may include vector unit circuitry, single instruction multiple data (SIMD) unit circuitry, load/store unit (LSU) circuitry, branch/jump unit circuitry, floating-point unit (FPU) circuitry, etc. The control unit circuitry 714 includes semiconductor-based circuits structured to control (e.g., coordinate) data movement within the corresponding core 702. The AL circuitry 716 includes semiconductor-based circuits structured to perform one or more mathematic and/or logic operations on the data within the corresponding core 702. The AL circuitry 716 of some examples performs integer based operations. In other examples, the AL circuitry 716 also performs floating point operations. In yet other examples, the AL circuitry 716 may include first AL circuitry that performs integer based operations and second AL circuitry that performs floating point operations. In some examples, the AL circuitry 716 may be referred to as an Arithmetic Logic Unit (ALU). The registers 718 are semiconductor-based structures to store data and/or instructions such as results of one or more of the operations performed by the AL circuitry 716 of the corresponding core 702. For example, the registers 718 may include vector register(s), SIMD register(s), general purpose register(s), flag register(s), segment register(s), machine specific register(s), instruction pointer register(s), control register(s), debug register(s), memory management register(s), machine check register(s), etc. The registers 718 may be arranged in a bank as shown in FIG. 7. Alternatively, the registers 718 may be organized in any other arrangement, format, or structure including distributed throughout the core 702 to shorten access time. The second bus 722 may be implemented by at least one of an I2C bus, a SPI bus, a PCI bus, or a PCIe bus.

Each core 702 and/or, more generally, the microprocessor 700 may include additional and/or alternate structures to those shown and described above. For example, one or more clock circuits, one or more power supplies, one or more power gates, one or more cache home agents (CHAs), one or more converged/common mesh stops (CMSs), one or more shifters (e.g., barrel shifter(s)) and/or other circuitry may be present. The microprocessor 700 is a semiconductor device fabricated to include many transistors interconnected to implement the structures described above in one or more integrated circuits (ICs) contained in one or more packages.

The programmable circuitry may include and/or cooperate with one or more accelerators (e.g., acceleration circuitry, hardware accelerators, etc.). In some examples, accelerators are implemented by logic circuitry to perform certain tasks more quickly and/or efficiently than can be done by a general purpose processor. Examples of accelerators include ASICs and FPGAs such as those discussed herein. A GPU, DSP, and/or other programmable device can also be an accelerator. Accelerators may be on-board the microprocessor 800, in the same chip package as the microprocessor 800 and/or in one or more separate packages from the microprocessor 800.

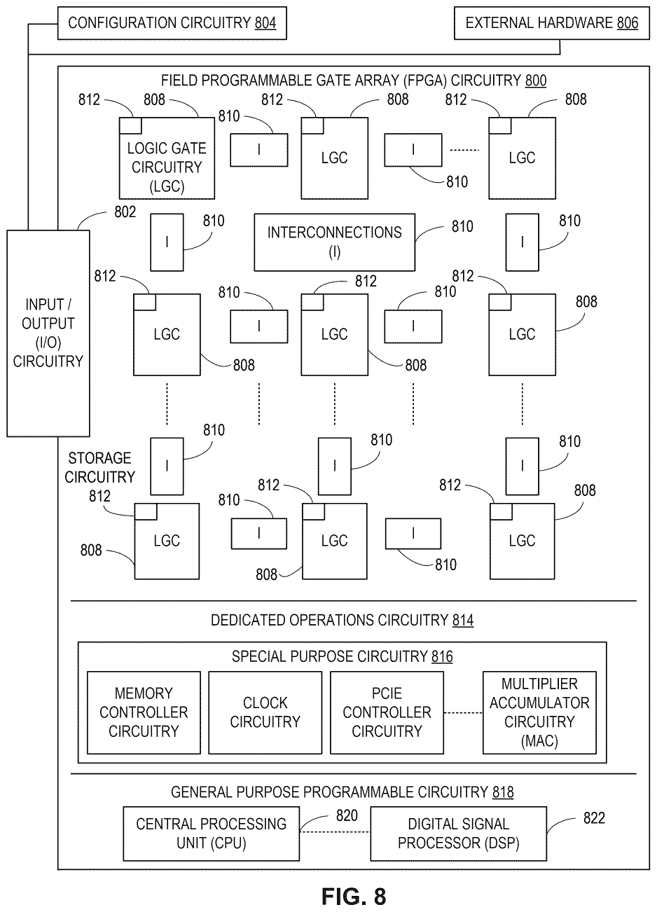

FIG. 8 is a block diagram of another example implementation of the programmable circuitry 612 of FIG. 6. In this example, the programmable circuitry 612 is implemented by FPGA circuitry 800. For example, the FPGA circuitry 800 may be implemented by an FPGA. The FPGA circuitry 800 can be used, for example, to perform operations that could otherwise be performed by the example microprocessor 700 of FIG. 7 executing corresponding machine readable instructions. However, once configured, the FPGA circuitry 800 instantiates the operations and/or functions corresponding to the machine readable instructions in hardware and, thus, can often execute the operations/functions faster than they could be performed by a general purpose microprocessor executing the corresponding software.

More specifically, in contrast to the microprocessor 700 of FIG. 7 described above (which is a general purpose device that may be programmed to execute some or all of the machine readable instructions represented by the flowchart of FIG. 5 but whose interconnections and logic circuitry are fixed once fabricated), the FPGA circuitry 800 of the example of FIG. 8 includes interconnections and logic circuitry that may be configured, structured, programmed, and/or interconnected in different ways after fabrication to instantiate, for example, some or all of the operations/functions corresponding to the machine readable instructions represented by the flowchart of FIG. 2. In particular, the FPGA circuitry 800 may be thought of as an array of logic gates, interconnections, and switches. The switches can be programmed to change how the logic gates are interconnected by the interconnections, effectively forming one or more dedicated logic circuits (unless and until the FPGA circuitry 800 is reprogrammed). The configured logic circuits enable the logic gates to cooperate in different ways to perform different operations on data received by input circuitry. Those operations may correspond to some or all of the instructions (e.g., the software and/or firmware) represented by the flowchart of FIG. 2. As such, the FPGA circuitry 800 may be configured and/or structured to effectively instantiate some or all of the operations/functions corresponding to the machine readable instructions of the flowchart of FIG. 2 as dedicated logic circuits to perform the operations/functions corresponding to those software instructions in a dedicated manner analogous to an ASIC. Therefore, the FPGA circuitry 800 may perform the operations/functions corresponding to the some or all of the machine readable instructions of FIG. 2 faster than the general purpose microprocessor can execute the same.

In the example of FIG. 8, the FPGA circuitry 800 is configured and/or structured in response to being programmed (and/or reprogrammed one or more times) based on a binary file. In some examples, the binary file may be compiled and/or generated based on instructions in a hardware description language (HDL) such as Lucid, Very High Speed Integrated Circuits (VHSIC) Hardware Description Language (VHDL), or Verilog. For example, a user (e.g., a human user, a machine user, etc.) may write code or a program corresponding to one or more operations/functions in an HDL; the code/program may be translated into a low-level language as needed; and the code/program (e.g., the code/program in the low-level language) may be converted (e.g., by a compiler, a software application, etc.) into the binary file. In some examples, the FPGA circuitry 800 of FIG. 8 may access and/or load the binary file to cause the FPGA circuitry 800 of FIG. 8 to be configured and/or structured to perform the one or more operations/functions. For example, the binary file may be implemented by a bit stream (e.g., one or more computer-readable bits, one or more machine-readable bits, etc.), data (e.g., computer-readable data, machine-readable data, etc.), and/or machine-readable instructions accessible to the FPGA circuitry 800 of FIG. 8 to cause configuration and/or structuring of the FPGA circuitry 800 of FIG. 8, or portion(s) thereof.

In some examples, the binary file is compiled, generated, transformed, and/or otherwise output from a uniform software platform utilized to program FPGAs. For example, the uniform software platform may translate first instructions (e.g., code or a program) that correspond to one or more operations/functions in a high-level language (e.g., C, C++, Python, etc.) into second instructions that correspond to the one or more operations/functions in an HDL. In some such examples, the binary file is compiled, generated, and/or otherwise output from the uniform software platform based on the second instructions. In some examples, the FPGA circuitry 800 of FIG. 8 may access and/or load the binary file to cause the FPGA circuitry 800 of FIG. 8 to be configured and/or structured to perform the one or more operations/functions. For example, the binary file may be implemented by a bit stream (e.g., one or more computer-readable bits, one or more machine-readable bits, etc.), data (e.g., computer-readable data, machine-readable data, etc.), and/or machine-readable instructions accessible to the FPGA circuitry 800 of FIG. 8 to cause configuration and/or structuring of the FPGA circuitry 800 of FIG. 8, or portion(s) thereof.

The FPGA circuitry 800 of FIG. 8, includes example input/output (I/O) circuitry 802 to obtain and/or output data to/from example configuration circuitry 804 and/or external hardware 806. For example, the configuration circuitry 804 may be implemented by interface circuitry that may obtain a binary file, which may be implemented by a bit stream, data, and/or machine readable instructions to configure the FPGA circuitry 800, or portion(s) thereof. In some such examples, the configuration circuitry 804 may obtain the binary file from a user, a machine (e.g., hardware circuitry (e.g., programmable or dedicated circuitry) that may implement an Artificial Intelligence/Machine Learning (AI/ML) model to generate the binary file), etc., and/or any combination(s) thereof. In some examples, the external hardware 806 may be implemented by external hardware circuitry. For example, the external hardware 806 may be implemented by the microprocessor 700 of FIG. 7. The FPGA circuitry 800 also includes an array of example logic gate circuitry 808, a plurality of example configurable interconnections 810, and example storage circuitry 812. The logic gate circuitry 808 and the configurable interconnections 810 are configurable to instantiate one or more operations that may correspond to at least some of the machine readable instructions of FIG. 2 and/or other desired operations. The logic gate circuitry 808 shown in FIG. 8 is fabricated in blocks or groups. Each block includes semiconductor-based electrical structures that may be configured into logic circuits. In some examples, the electrical structures include logic gates (e.g., And gates, Or gates, Nor gates, etc.) that provide basic building blocks for logic circuits. Electrically controllable switches (e.g., transistors) are present within each of the logic gate circuitry 808 to enable configuration of the electrical structures and/or the logic gates to form circuits to perform desired operations/functions. The logic gate circuitry 808 may include other electrical structures such as look-up tables (LUTs), registers (e.g., flip-flops or latches), multiplexers, etc.

The configurable interconnections 810 of the illustrated example are conductive pathways, traces, vias, or the like that may include electrically controllable switches (e.g., transistors) whose state can be changed by programming (e.g., using an HDL instruction language) to activate or deactivate one or more connections between one or more of the logic gate circuitry 808 to program desired logic circuits.

The storage circuitry 812 of the illustrated example is structured to store result(s) of the one or more of the operations performed by corresponding logic gates. The storage circuitry 812 may be implemented by registers or the like. In the illustrated example, the storage circuitry 812 is distributed amongst the logic gate circuitry 808 to facilitate access and increase execution speed.

The example FPGA circuitry 800 of FIG. 8 also includes example Dedicated Operations Circuitry 814. In this example, the Dedicated Operations Circuitry 814 includes special purpose circuitry 816 that may be invoked to implement commonly used functions to avoid the need to program those functions in the field. Examples of such special purpose circuitry 816 include memory (e.g., DRAM) controller circuitry, PCIe controller circuitry, clock circuitry, transceiver circuitry, memory, and multiplier-accumulator circuitry. Other types of special purpose circuitry may be present. In some examples, the FPGA circuitry 800 may also include example general purpose programmable circuitry 818 such as an example CPU 820 and/or an example DSP 822. Other general purpose programmable circuitry 818 may additionally or alternatively be present such as a GPU, an XPU, etc., that can be programmed to perform other operations.

Although FIGS. 7 and 8 illustrate two example implementations of the programmable circuitry 612 of FIG. 6, many other approaches are contemplated. For example, FPGA circuitry may include an on-board CPU, such as one or more of the example CPU 720 of FIG. 7. Therefore, the programmable circuitry 612 of FIG. 6 may additionally be implemented by combining at least the example microprocessor 700 of FIG. 7 and the example FPGA circuitry 800 of FIG. 8. In some such hybrid examples, one or more cores 702 of FIG. 7 may execute a first portion of the machine readable instructions represented by the flowchart of FIG. 5 to perform first operation(s)/function(s), the FPGA circuitry 800 of FIG. 8 may be configured and/or structured to perform second operation(s)/function(s) corresponding to a second portion of the machine readable instructions represented by the flowchart of FIG. 5, and/or an ASIC may be configured and/or structured to perform third operation(s)/function(s) corresponding to a third portion of the machine readable instructions represented by the flowchart of FIG. 5.

It should be understood that some or all of the circuitry of FIG. 2 may, thus, be instantiated at the same or different times. For example, same and/or different portion(s) of the microprocessor 700 of FIG. 7 may be programmed to execute portion(s) of machine-readable instructions at the same and/or different times. In some examples, same and/or different portion(s) of the FPGA circuitry 800 of FIG. 8 may be configured and/or structured to perform operations/functions corresponding to portion(s) of machine-readable instructions at the same and/or different times.

In some examples, some or all of the circuitry of FIG. 2 may be instantiated, for example, in one or more threads executing concurrently and/or in series. For example, the microprocessor 700 of FIG. 7 may execute machine readable instructions in one or more threads executing concurrently and/or in series. In some examples, the FPGA circuitry 800 of FIG. 8 may be configured and/or structured to carry out operations/functions concurrently and/or in series. Moreover, in some examples, some or all of the circuitry of FIG. 2 may be implemented within one or more virtual machines and/or containers executing on the microprocessor 700 of FIG. 7.

In some examples, the programmable circuitry 612 of FIG. 6 may be in one or more packages. For example, the microprocessor 700 of FIG. 7 and/or the FPGA circuitry 800 of FIG. 8 may be in one or more packages. In some examples, an XPU may be implemented by the programmable circuitry 612 of FIG. 6, which may be in one or more packages. For example, the XPU may include a CPU (e.g., the microprocessor 700 of FIG. 7, the CPU 820 of FIG. 8, etc.) in one package, a DSP (e.g., the DSP 822 of FIG. 8) in another package, a GPU in yet another package, and an FPGA (e.g., the FPGA circuitry 800 of FIG. 8) in still yet another package.

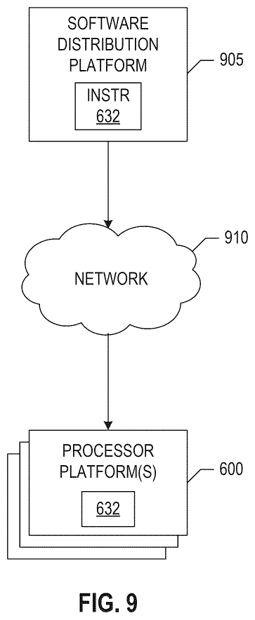

A block diagram illustrating an example software distribution platform 905 to distribute software such as the example machine readable instructions 632 of FIG. 6 (corresponding the mixed reality program 216) to other hardware devices (e.g., hardware devices owned and/or operated by third parties from the owner and/or operator of the software distribution platform) is illustrated in FIG. 9. The example software distribution platform 905 may be implemented by any computer server, data facility, cloud service, etc., capable of storing and transmitting software to other computing devices. The third parties may be customers of the entity owning and/or operating the software distribution platform 905. For example, the entity that owns and/or operates the software distribution platform 905 may be a developer, a seller, and/or a licensor of software such as the example machine readable instructions 632 of FIG. 6. The third parties may be consumers, users, retailers, OEMs, etc., who purchase and/or license the software for use and/or re-sale and/or sub-licensing. In the illustrated example, the software distribution platform 905 includes one or more servers and one or more storage devices. The storage devices store the machine readable instructions 632, which may correspond to the example machine readable instructions 500 of FIG. 5, as described above. The one or more servers of the example software distribution platform 905 are in communication with an example network 910, which may correspond to any one or more of the Internet and/or any of the example networks described above. In some examples, the one or more servers are responsive to requests to transmit the software to a requesting party as part of a commercial transaction. Payment for the delivery, sale, and/or license of the software may be handled by the one or more servers of the software distribution platform and/or by a third party payment entity. The servers enable purchasers and/or licensors to download the machine readable instructions 632 from the software distribution platform 905. For example, the software, which may correspond to the example machine readable instructions 500 of FIG. 5, may be downloaded to the example processor platform 600, which is to execute the machine readable instructions 632 to implement the mixed reality program 216. In some examples, one or more servers of the software distribution platform 905 periodically offer, transmit, and/or force updates to the software (e.g., the example machine readable instructions 632 of FIG. 6) to ensure improvements, patches, updates, etc., are distributed and applied to the software at the end user devices.

From the foregoing, it will be appreciated that example systems, methods, apparatus, and articles of manufacture have been disclosed that help calm or reduce anxiety of a person during an anxiety-inducing process or situation such as, for example a medical procedure, such as a blood collection operation. Examples disclosed herein leverage immersive technology to entertain and/or otherwise direct the person's focus away from the blood collection process. This helps reduce the person's fear or anxiety and, thus, the person is more comfortable during the anxiety-inducing situation. The example methods and apparatus can also reduce pain or the perception of pain by distracting and/or entertaining the person during the blood collection process. As such, the examples disclosed herein can lead to increase donor engagement, satisfaction, and retention (e.g., repeat donations). Further, by reducing fear and/or anxiety, the example methods and apparatus can help in increasing donations from certain demographics that have historically or statistically lower donation rates.

Examples and example combinations disclosed herein include the following:
Example 1 is a non-transitory machine-readable medium comprising instructions that, when executed, cause programmable circuitry to: present a virtual avatar on a display of a headset worn by a patient during a blood collection process. The display is at least partially transparent to enable the patient to view real-world surroundings through the display and such that the virtual avatar appears within the real-world surroundings. The display is at least partially transparent to enable a medical professional to monitor an eye of the patient during the blood collection process. The virtual avatar is to distract the patient and reduce anxiety in the patient. The instructions also cause the programmable circuitry to detect objects and surfaces in the real-world surroundings, create a three-dimensional (3D) model of the real-world surroundings, present a first digital image on the display, a position of the first digital image based on the 3D model such that the first digital image appears on one or more of the objects or surfaces in the real-world surroundings, determine a focal point of the patient based on a gaze direction of the eye of the patient, determine an amount of time the patient holds the focal point, compare the amount of time to a threshold period of time, and, in response to the amount of time satisfying the threshold, present a second digital image on the display in place of the first digital image. The position of the second digital image on the display is based on the 3D model such that the second digital image appears on one or more of the objects or surfaces in the real-world surroundings.
Example 2 includes the machine-readable medium of Example 1, wherein the instructions, when executed, cause the programmable circuitry to present the virtual avatar on a first portion of the display and present the first digital image on a second portion of the display. The second portion is different than the first portion.
Example 3 includes the machine-readable medium of Examples 1 or 2, wherein the instructions, when executed, cause the programmable circuitry to animate a transformation of the first digital image to the second digital image.
Example 4 includes the machine-readable medium of Examples 1-3, wherein the instructions, when executed, cause the programmable circuitry to identify flatness of the objects and surfaces in the 3D model and identify the position of the second digital image based on the flatness.
Example 5 includes the machine-readable medium of any of Examples 1-4, wherein the instructions when executed, cause the programmable circuitry to present an animation of a moving object on the display and appearing in the real-world surroundings and prompt the patient to follow the moving object with eye gaze.
Example 6 includes the machine-readable medium of any of Examples 1-5, wherein the instructions, when executed, cause the programmable circuitry to track eye gaze of the patient, assess an activity level of the patient based on the eye gaze, and automatically present a sequence of additional digital images on the display when the activity level does not satisfy a threshold level of activity. The threshold level of activity is based on time.
Example 7 includes the machine-readable medium of any of Examples 1-6, wherein the instructions cause the programmable circuitry to present the virtual avatar in different positions on the display such that the virtual avatar appears to move around the real-world surroundings to train the patient with eye gaze control.
Example 8 includes the machine-readable medium of any of Examples 1-7, wherein the instructions cause the programmable circuitry to present audio instructions via a speaker.
Example 9 is a mixed reality headset to be used during a blood collection process. The mixed reality headset comprises a headband to be placed around a head of a person and a visor carried by the headband. The visor is to be disposed over eyes of the person wearing the headband. The visor is at least partially transparent to enable the person to see real-world surroundings and to enable a medical professional to monitor the eyes of the person. The mixed reality headset also includes a display device to display digital content on the visor, memory, and programmable circuitry to execute instructions to track objects and surfaces in the real-world surroundings, create a three-dimensional (3D) model of the real-world surroundings, cause the display device to present a first virtual object on the visor such that the first virtual object appears in the real-world surroundings, track a focal point of the person, determine an amount of time the person holds the focal point, compare the amount of time to a threshold time, and, in response to the amount of time satisfying the threshold time, cause the display device to present a transformation of the first virtual object on the visor.
Example 10 includes the mixed reality headset of Example 9, wherein the programmable circuitry is to cause the display device to present an avatar on the visor. The avatar is to distract the person and reduce anxiety in the person.
Example 11 includes the mixed reality headset of Example 10, further including a speaker. The programmable circuitry is to cause the speaker to provide audible instructions coordinated with the avatar.
Example 12 is a method of reducing anxiety in a person during a blood collection process. The method comprises providing a mixed reality headset to a person prior to collecting blood from the person. The mixed reality headset includes glasses and a display device to display digital content on the glasses. The glasses enable a medical professional to monitor eyes of the person during the blood collection process. The method also includes initiating a mixed reality program on the mixed reality headset. The mixed reality program is to reduce anxiety by causing the display device to display a mixed reality environment with one or more virtual objects on the glasses such that the virtual objects appear to be located in a real-world environment of the person, determining a gaze direction of the eyes of the person, and causing a change in one or more of the virtual objects in the mixed reality environment based on the gaze direction of the eyes of the person to enable the person to control the one or more of the virtual objects. The method also includes collecting blood from the person while the person is exposed to the mixed reality program.
Example 13 includes the method of Example 12, wherein the mixed reality program is to reduce anxiety by further: detecting, via a sensor on the headset, objects and surfaces in the real-world environment; creating a three-dimensional (3D) model of the real-world environment; determining a direction of orientation of the headset in the real-world environment; and causing the display device to display the virtual objects on the glasses based on the 3D model and the direction of orientation of the headset such that the virtual objects appear fixed relative to one or more of the objects or surfaces in the real-world environment.
Example 14 includes the method of Example 13, wherein the virtual objects include a first virtual object displayed on a first portion of the glasses to appear on a surface in the real-world environment.
Example 15 includes the method of Example 14, wherein the mixed reality program is to reduce anxiety by further: determining an amount of time the person holds the gaze direction on a portion of the glasses containing the first virtual object; and comparing the amount of time to a threshold period of time.
Example 16 includes the method of Example 15, wherein the mixed reality program is to reduce anxiety by further, in response to the amount of time satisfying the threshold, causing the display to present a second virtual object image on the glasses in place of the first virtual object.
Example 17 includes the method of Examples 15 or 16, wherein the mixed reality program is to reduce anxiety by further, in response to the amount of time satisfying the threshold, causing an animation of the first virtual object into a second virtual object.
Example 18 includes the method of any of Examples 12-17, wherein the mixed reality program is to reduce anxiety by further causing the display device to present a virtual avatar on the glasses, the virtual avatar to provide instructions to the person for interacting with the mixed reality environment.
Example 19 includes the method of any of Examples 12-18, wherein the mixed reality program is to reduce anxiety by further activating a speaker on the mixed reality headset to provide audio instructions to the person.
Example 20 includes the method of any of Examples 12-19, wherein the person does not interact with the mixed reality environment via a hand-held device.
Example 21 is a method comprising providing a headset to a person, placing the headset on a head of the person, initiating a mixed reality program on the headset, and performing a blood collection process on the person while the person is exposed to the mixed reality program.
Example 22 includes the method of Example 21, wherein the mixed reality program is interactive.
Example 23 includes the method of Example 22, wherein the person interacts with the mixed reality program via gaze control.
Example 24 includes the method of any of Examples 21-23, wherein the mixed reality program displays digital content including images of plants.
Example 25 includes the method of any of Examples 21-24, wherein the mixed reality program is initiated prior to starting the blood collection process.
Example 26 includes the method of any of Examples 21-25, monitoring the person's eyes via transparent glass on the headset.
Example 27 is a non-transitory machine readable storage medium comprising instructions that, when executed, cause programmable circuitry of a headset to at least display digital content on the headset to a person wearing the headset during a blood collection process, the digital content to create a mixed reality environment.
Example 28 includes the non-transitory machine readable storage medium of Example 27, wherein the digital content is interactive.
Example 29 includes the non-transitory machine readable storage medium of Example 28, wherein the instructions cause the programmable circuitry to determine a direction of the person's gaze and perform an action based on the person's gaze.
Example 30 is a headset comprising: glasses, a display device to display digital content on the glasses, memory, a plurality of mixed reality programs in the memory, and programmable circuitry to execute instructions to: identify a duration of time of a medical procedure, select one of the plurality of mixed reality programs based on the duration of time, present the selected mixed reality program on the glasses, determine a gaze of a person wearing the headset during presentation of the selected mixed reality program; and change the presentation of the mixed reality program based on the gaze.
Example 31 includes the headset of Example 30, wherein the glass is transparent to enable a phlebotomist to monitor the person's eyes.
Example 32 includes the headset of Examples 30 or 31, wherein the selected mixed reality program is interactive.
Example 33 includes the headset of Example 32, wherein digital content in the mixed reality program is controllable via gaze control.
Example 34 includes the headset of claim 33, wherein the headset includes an eye tracking sensor.
Example 35 is a method comprising exposing a person to a mixed reality environment during a medical procedure.

The following claims are hereby incorporated into this Detailed Description by this reference. Although certain example systems, methods, apparatus, and articles of manufacture have been disclosed herein, the scope of coverage of this patent is not limited thereto. On the contrary, this patent covers all systems, methods, apparatus, and articles of manufacture fairly falling within the scope of the claims of this patent.

What is claimed is:

1. A non-transitory machine-readable medium comprising instructions that, when executed, cause programmable circuitry to:
present a virtual avatar on a display of a headset worn by a patient during a blood collection process, the display being at least partially transparent to enable the patient to view real-world surroundings through the display and such that the virtual avatar appears within the real-world surroundings, the display being at least partially transparent to enable a medical professional to monitor an eye of the patient during the blood collection process, and the virtual avatar to distract the patient and reduce anxiety in the patient;
detect objects and surfaces in the real-world surroundings;
create a three-dimensional (3D) model of the real-world surroundings;
present a first digital image on the display, a position of the first digital image based on the 3D model such that the first digital image appears on one or more of the objects or surfaces in the real-world surroundings;
determine a focal point of the patient based on a gaze direction of the eye of the patient;
determine an amount of time the patient holds the focal point;
compare the amount of time to a threshold period of time; and
in response to the amount of time satisfying the threshold, present a second digital image on the display in place of the first digital image, the position of the second digital image on the display based on the 3D model such that the second digital image appears on one or more of the objects or surfaces in the real-world surroundings.

2. The machine-readable medium of claim 1, wherein the instructions, when executed, cause the programmable circuitry to present the virtual avatar on a first portion of the display and present the first digital image on a second portion of the display, the second portion different than the first portion.

3. The machine-readable medium of claim 1, wherein the instructions, when executed, cause the programmable circuitry to animate a transformation of the first digital image to the second digital image.

4. The machine-readable medium of claim 1, wherein the instructions, when executed, cause the programmable circuitry to:
identify flatness of the objects and surfaces in the 3D model; and
identify the position of the second digital image based on the flatness.

5. The machine-readable medium of claim 1, wherein the instructions when executed, cause the programmable circuitry to:
present an animation of a moving object on the display and appearing in the real-world surroundings; and
prompt the patient to follow the moving object with eye gaze.

6. The machine-readable medium of claim 1, wherein the instructions, when executed, cause the programmable circuitry to:
track eye gaze of the patient;
assess an activity level of the patient based on the eye gaze; and
automatically present a sequence of additional digital images on the display when the activity level does not satisfy a threshold level of activity, the threshold level of activity based on time.

7. The machine-readable medium of claim 1, wherein the instructions cause the programmable circuitry to present the virtual avatar in different positions on the display such that the virtual avatar appears to move around the real-world surroundings to train the patient with eye gaze control.

8. The machine-readable medium of claim 1, wherein the instructions cause the programmable circuitry to present audio instructions via a speaker.

9. A mixed reality headset to be used during a blood collection process, the mixed reality headset comprising:
a headband to be placed around a head of a person;
a visor carried by the headband, the visor to be disposed over eyes of the person wearing the headband, the visor being at least partially transparent to enable the person to see real-world surroundings and to enable a medical professional to monitor the eyes of the person;
a display device to display digital content on the visor;
memory; and
programmable circuitry to execute instructions to:
track objects and surfaces in the real-world surroundings;
create a three-dimensional (3D) model of the real-world surroundings;
cause the display device to present a first virtual object on the visor such that the first virtual object appears in the real-world surroundings;
track a focal point of the person;
determine an amount of time the person holds the focal point;
compare the amount of time to a threshold time; and
in response to the amount of time satisfying the threshold time, cause the display device to present a transformation of the first virtual object on the visor.

10. The mixed reality headset of claim 9, wherein the programmable circuitry is to cause the display device to present an avatar on the visor, the avatar to distract the person and reduce anxiety in the person.

11. The mixed reality headset of claim 10, further including a speaker, the programmable circuitry to cause the speaker to provide audible instructions coordinated with the avatar.

12. A method of reducing anxiety in a person during a blood collection process, the method comprising:
providing a mixed reality headset to a person prior to collecting blood from the person, the mixed reality headset including glasses and a display device to display digital content on the glasses, the glasses to enable a medical professional to monitor eyes of the person during the blood collection process;
initiating a mixed reality program on the mixed reality headset, the mixed reality program to reduce anxiety by:
causing the display device to display a mixed reality environment with one or more virtual objects on the glasses such that the virtual objects appear to be located in a real-world environment of the person;
determining a gaze direction of the eyes of the person; and
causing a change in one or more of the virtual objects in the mixed reality environment based on the gaze direction of the eyes of the person to enable the person to control the one or more of the virtual objects; and
collecting blood from the person while the person is exposed to the mixed reality program.

13. The method of claim 12, wherein the mixed reality program is to reduce anxiety by further:
detecting, via a sensor on the headset, objects and surfaces in the real-world environment;
creating a three-dimensional (3D) model of the real-world environment;
determining a direction of orientation of the headset in the real-world environment; and
causing the display device to display the virtual objects on the glasses based on the 3D model and the direction of orientation of the headset such that the virtual objects appear fixed relative to one or more of the objects or surfaces in the real-world environment.

14. The method of claim 13, wherein the virtual objects include a first virtual object displayed on a first portion of the glasses to appear on a surface in the real-world environment.

15. The method of claim 14, wherein the mixed reality program is to reduce anxiety by further:
determining an amount of time the person holds the gaze direction on a portion of the glasses containing the first virtual object; and
comparing the amount of time to a threshold period of time.

16. The method of claim 15, wherein the mixed reality program is to reduce anxiety by further, in response to the amount of time satisfying the threshold, causing the display to present a second virtual object image on the glasses in place of the first virtual object.

17. The method of claim 15, wherein the mixed reality program is to reduce anxiety by further, in response to the amount of time satisfying the threshold, causing an animation of the first virtual object into a second virtual object.

18. The method of claim 12, wherein the mixed reality program is to reduce anxiety by further causing the display device to present a virtual avatar on the glasses, the virtual avatar to provide instructions to the person for interacting with the mixed reality environment.

19. The method of claim 12, wherein the mixed reality program is to reduce anxiety by further activating a speaker on the mixed reality headset to provide audio instructions to the person.

20. The method of claim 12, wherein the person does not interact with the mixed reality environment via a hand-held device.
